(19) United States
(12) Patent Application Publication
(10) Pub. No.: US 2013/0259926 A1
(43) Pub. Date: Oct. 3, 2013

(54) BI-FUNCTIONAL shRNA TARGETING MESOTHELIN AND USES THEREOF

(71) Applicants: GRADALIS, INC., Carrollton, TX (US); BAYLOR COLLEGE OF MEDICINE, Houston, TX (US)

(72) Inventors: Donald Rao, Dallas, TX (US); Zhaohui Wang, Grapevine, TX (US); John J. Nemunaitis, Cedar Hill, TX (US); Neil Senzer, Dallas, TX (US); Qizhi Yao, Houston, TX (US); Changyi (Johnny) Chen, Houston, TX (US); F. Charles Brunicardi, Pacific Palisades, CA (US)

(21) Appl. No.: 13/852,446

(22) Filed: Mar. 28, 2013

Related U.S. Application Data
(60) Provisional application No. 61/616,824, filed on Mar. 28, 2012.

(51) Int. Cl. C12N 15/113

(52) U.S. Cl. CPC C12N 15/1138
USPC 424/450; 536/24.5; 435/320.1; 514/44 A; 435/455

(57) ABSTRACT

The present invention includes bifunctional shRNAs capable of reducing an expression of a Mesothelin gene; wherein at least one target site sequence of the bifunctional RNA molecule is located within the Mesothelin, wherein the bifunctional RNA molecule is capable of activating a cleavage-dependent and a cleavage-independent RNA-induced silencing complex for reducing the expression level of Mesothelin.

### CROSS-REFERENCE TO RELATED APPLICATIONS

This application claims priority to U.S. Provisional Application Ser. No. 61/616,824, filed Mar. 28, 2012, the entire contents of which are incorporated herein by reference.

### TECHNICAL FIELD OF THE INVENTION

The present invention relates in general to the field of treatment of cancer, and more particularly, to bi-functional shRNA designs to knockdown the expression of Mesothelin (MSLN).

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with bi-functional shRNA targeting Mesothelin and uses thereof, as well as treatment for prostate cancer.

U.S. Pat. No. 7,745,159 entitled “Methods and compositions for diagnosing carcinomas”, issued to Scholler et al (2004) is directed to compositions and methods for the detection of a malignant condition, and relates to the discovery of soluble forms of mesothelin polypeptides, including mesothelin related antigen (MRA). In particular the invention provides a nucleic acid sequence encoding MRA and an MRA variant. The patent also provides a method of screening for the presence of a malignant condition in a subject by detecting reactivity of an antibody specific for a mesothelin polypeptide with a molecule naturally occurring in soluble form in a sample from such a subject, and by hybridization screening using an MRA nucleotide sequence, as well as other related advantages.

U.S. Pat. No. 6,083,502 entitled “Mesothelium antigen and methods and kits for targeting it” issued to Pastan et al (2000) relates to the discovery of Mesothelin as a differentiation antigen, which is associated with mesotheliomas and ovarian cancers. The patent includes uses for the amino acid and nucleic acid sequences for mesothelin, recombinant cells expressing it, methods for targeting and/or inhibiting the growth of cells bearing mesothelin, methods for detecting the antigen and its expression level as an indication of the presence of tumor cells, and kits for such detection.

United States Patent Application 20110236385, entitled “Blocking mesothelin peptide fragments”, filed by Ho et al (2008) provides mesothelin peptide fragments corresponding to the CA125 binding site of mesothelin. The peptide fragments are said to find use in disrupting the binding interaction between mesothelin and CA 125, for example, in the treatment and prevention of cancers that require the interaction of mesothelin and CA125 for growth, progression and/or metastasis.

### SUMMARY OF THE INVENTION

The present invention includes bifunctional shRNAs capable of reducing an expression of a Mesothelin gene; wherein at least one target site sequence of the bifunctional RNA molecule is located within the Mesothelin gene; wherein the bifunctional RNA molecule is capable of activating a cleavage-dependent and a cleavage-independent RNA-induced silencing complex for reducing the expression level of Mesothelin. In various aspects, the bifunctional shRNA comprises at least one sequence defined by SEQ ID NO: 6 or SEQ ID NO: 8. In certain aspects, at least one target site sequence is within a human Mesothelin gene cDNA sequence (SEQ ID NO: 1 or 2); and/or is SEQ ID NO: 3 or SEQ ID NO: 4. The invention also includes embodiments of expression vectors comprising a promoter and a nucleic acid insert operably linked to the promoter, wherein the insert encodes one or more shRNA capable of inhibiting an expression of at least one target gene that is a Mesothelin gene via RNA interference, wherein the one or more shRNA comprise a bifunctional RNA molecule that activates a cleavage-dependent and a cleavage-independent RNA-induced silencing complex for reducing the expression level of Mesothelin. The target gene sequence may comprise SEQ ID NO: 1 or 2. In certain aspects, the sequence arrangement for the shRNA may comprise a 5′ stem arm-19 nucleotide target, which is Mesothelin-TA-15 nucleotide loop-19 nucleotide target complementary sequence-3′stem arm-Spacer-5′ stem arm-19 nucleotide target variant-TA-15 nucleotide loop-19 nucleotide target complementary sequence-3′stem arm. The nucleic acid insert comprises at least one sequence selected from SEQ ID NO: 6 or SEQ ID NO: 8. In certain aspects, at least one shRNA may have a target site sequence that is within a Mesothelin gene cDNA sequence. In certain aspects, the expression vector of claim 6 may be selected from the group consisting of SEQ ID NO: 9 or SEQ ID NO: 10. The invention also includes embodiments for a therapeutic delivery system comprising a therapeutic agent carrier, and an expression vector comprising a promoter and a nucleic acid insert operably linked to the promoter, the nucleic acid insert encoding one or more short hairpin RNA (shRNA) capable inhibiting an expression of a target gene sequence that is Mesothelin gene via RNA interference; wherein the one or more shRNA comprise a bifunctional RNA molecule that activates a cleavage-dependent and a cleavage-independent RNA-induced silencing complex for reducing the expression level of Mesothelin. The therapeutic agent carrier may be a compacted DNA nanoparticle, and the DNA nanoparticle may be compacted with one or more polycations. In certain aspects, the one or more polycations may be a 10 kDA polyethylene glycol (PEG)-substituted cysteine-lysine 3-mer peptide (CK30PEG10k), and the compacted DNA nanoparticles may be further encapsulated in a liposome. In some aspects, the liposome may be bilamellar invaginated vesicle (BIV); the liposome may be a reversibly masked liposome. The therapeutic agent carrier is a liposome. In certain aspects, the target gene sequence may comprise SEQ ID NO: 3 or 4, and/or the nucleic acid insert may comprise at least one of the sequences selected from SEQ ID NO: 6, SEQ ID NO: 8, SEQ ID NO: 9, or SEQ ID NO: 10. Other embodiments of the invention include methods to deliver one or more shRNAs to a target tissue expressing a Mesothelin gene comprising the steps of preparing an expression vector comprising a promoter and a nucleic acid insert operably linked to the promoter that encodes the one or more shRNA, wherein the one or more shRNA comprise a bifunctional RNA molecule that activates a cleavage-dependent and a cleavage-independent RNA-induced silencing complex for reducing the expression level of Mesothelin, combining the expression vector with a therapeutic agent carrier, wherein the therapeutic agent carrier comprises a liposome, and administering a therapeutically effective amount of the expression vector and therapeutic agent carrier complex to a patient in need thereof. The therapeutic agent carrier may be a compacted DNA nanoparticle. The DNA nanoparticle may be compacted with one or more polycations, wherein the one or more polycations comprise a 10 kDA polyethylene glycol (PEG)-substituted cysteine-lysine 3-mer peptide (CK30PEG10k) or a 30-mer lysine condensing peptide. IN certain aspects of the invention, the compacted DNA nanoparticles may be further encapsulated in a liposome, wherein the liposome is a bilamellar invaginated vesicle (BIV) and is decorated with one or more “smart” receptor targeting moieties, and the one or more “smart” receptor targeting moieties may be small molecule bivalent beta-turn mimics. In certain aspects, the liposome may be a reversibly masked liposome, and the liposome may be a bilamellar invaginated vesicle (BIV). In certain aspects, the liposome is a reversibly masked liposome, and the one or more “smart” receptor targeting moieties may be small molecule bivalent beta-turn mimics. In certain aspects, the vector is selected from one of SEQ ID NO: 9 or SEQ ID NO: 10, and/or the nucleic acid insert comprises a sequence selected from SEQ ID NO: 6, or SEQ ID NO: 8. Certain embodiments of the invention include methods to inhibit an expression of a Mesothelin gene in one or more target cells comprising the steps of selecting the one or more target cells, and transfecting the target cell with a vector that expresses one or more short hairpin RNA (shRNAs) capable of inhibiting an expression of a Mesothelin gene in the one or more target cells via RNA interference, wherein the one or more shRNA comprise a bifunctional RNA molecule that activates a cleavage-dependent and a cleavage-independent RNA-induced silencing complex for reducing the expression level of Mesothelin, wherein the shRNA may incorporate siRNA (cleavage-dependent) and miRNA (cleavage-independent) motifs. In certain embodiments, the invention includes methods of suppressing a tumor cell growth in a human subject comprising the steps of identifying the human subject in need for suppression of the tumor cell growth; and administering an expression vector in a therapeutic agent carrier complex to the human subject in an amount sufficient to suppress the tumor cell growth, wherein the expression vector expresses one or more shRNA capable inhibiting an expression of a target gene that is Mesothelin in the one or more target cells via RNA interference; wherein the one or more shRNA comprise a bifunctional RNA molecule that activates a cleavage-dependent and a cleavage-independent RNA-induced silencing complex for reducing the expression level of the target gene; wherein the inhibition results in an apoptosis, an arrested proliferation, or a reduced invasiveness of the tumor cells. The therapeutic agent carrier may comprise a bilamellar invaginated vesicle (BIV), and the therapeutic agent carrier may comprise one or more “smart” receptor targeting moieties are small molecule bivalent beta-turn mimics. In certain aspects of the invention, administering is selected from the group consisting of subcutaneous, intravenous, intraperitoneal, intramuscular, and intravenous injection; and/or administering may comprise intratumoral injection. Administering may comprise injecting with a DNA:lipoplex. In certain aspects, tumor cell growth expresses Mesothelin, and/or may be human pancreatic ductal adenocarcinoma. In certain aspects the tumor cell growth may be selected from the group consisting of insulinoma, mesothelioma, ovarian cancer, and pancreatic cancer; and/or the tumor cell growth is selected from the group consisting of epithelial mesothelioma, squamous cell carcinoma, head and neck cancer, lung cancer, cervix cancer, esophagus cancer, and gastric adenocarcinoma.

### BRIEF DESCRIPTION OF THE DRAWINGS

For a more complete understanding of the features and advantages of the present invention, reference is now made to the detailed description of the invention along with the accompanying figures and in which:

FIG. 1 illustrates the predicted stem-loop structure of MSLN-1.

FIG. 2 shows a plasmid map of bi-sh-MSLN-1.

FIG. 3 illustrates the predicted stem-loop structure of MSLN-2.

FIG. 4 shows a plasmid map of bi-sh-MSLN-2.

FIG. 5 depicts western blot data showing that sh33, which is pGBI-33 (bi-shRNA-MSLN-1), efficiently knocks-down MSLN in Capan2 cells, a pancreatic cancer cell line.

FIG. 6 shows various levels of MSLN expression in MIA-MSLN (forced MSLN expression stable cell line), Capan-2 (endogenous MSLN high expression cell line), and MIA-V (expression vector control stable cell line) by using western blot.

FIG. 7 shows the efficiency of transfection is more than 90% when MIA-MSLN or Capan-2 cells were transfected with sh33 (bi-shRNA against MSLN), sh02 (positive control MSLN knock-down plasmid obtained from a commercial source), or pUMVC3 (empty vector control).

FIG. 8 at 72 h post transfection, RNA and protein were extracted from the transfected cells, qRT-PCR was run to compare the MSLN mRNA expression level changes upon different plasmids transfection in both cell lines and western immunoblot was run to compare MSLN protein levels. This is a western blot that shows knockdown effect in the MSLN protein expression level in both MIA-MSLN and Capan-2 cells.

FIGS. 9A and 9B show the knockdown effect of the present invention monitored at mRNA level in both MIA-MSLN and Capan-2 cells.

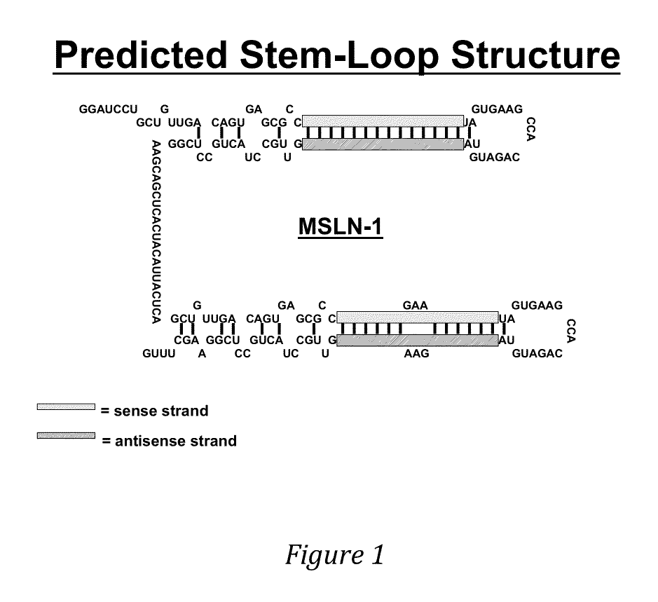

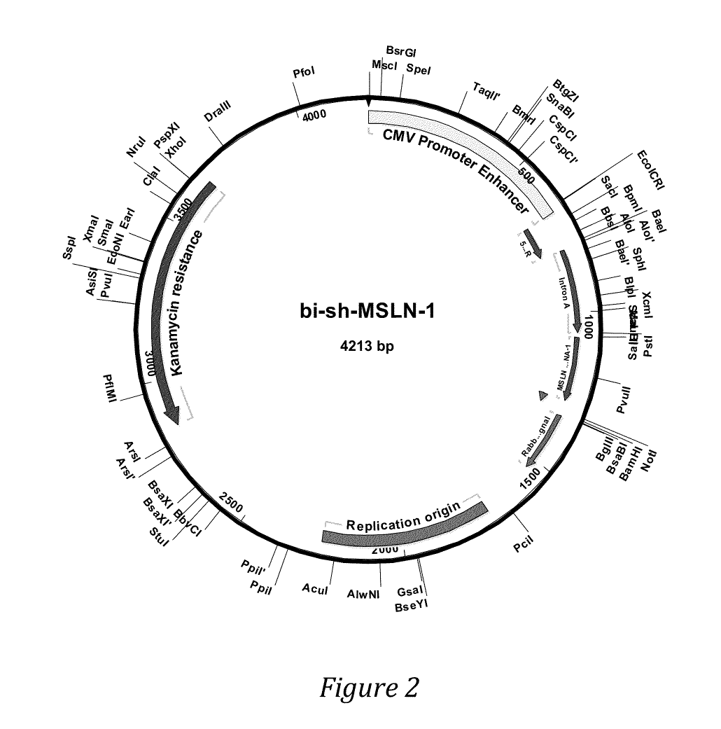

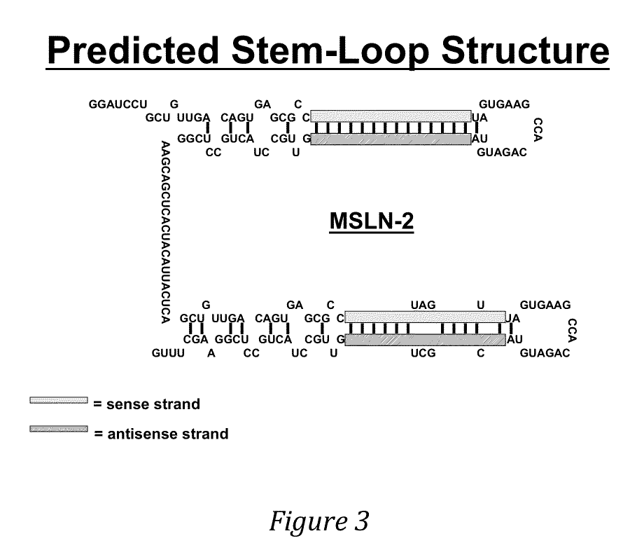

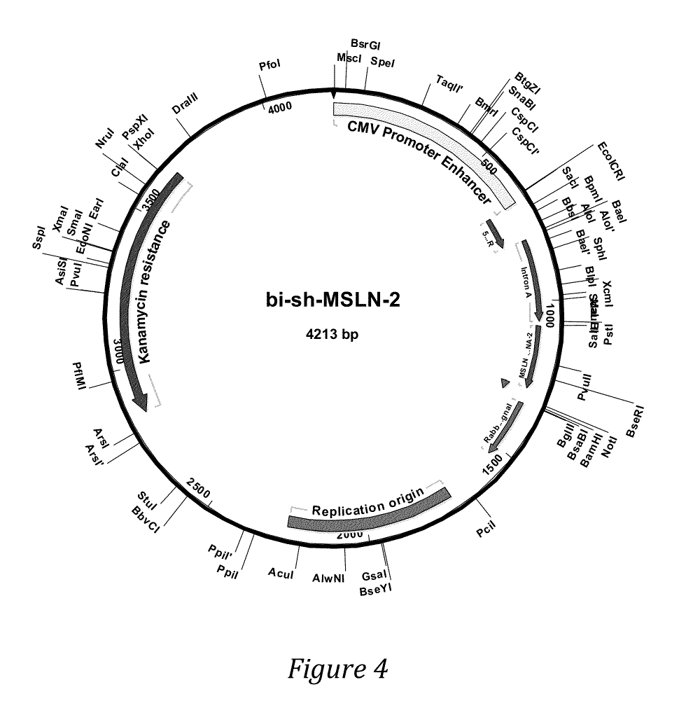

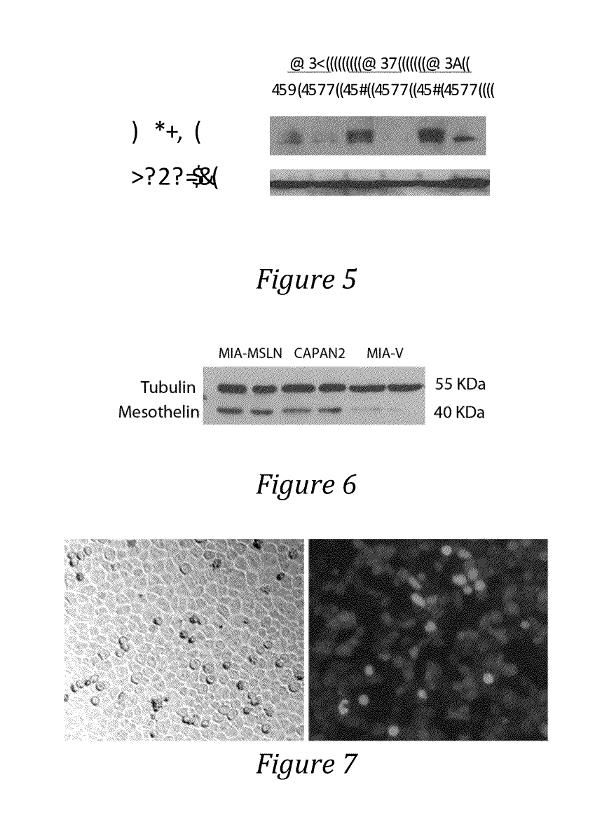

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Mesothelin (MSLN) a cell surface antigen that was originally identified using an antibody that reacts with ovarian cancers and mesotheliomas. Although the function of mesothelin is unknown, it may play a role in cellular adhesion and is present on mesothelium, mesotheliomas, and ovarian cancers. The mesothelin gene encodes a preproprotein that is cleaved into megakaryocyte potentiating factor and mesothelin. The present inventors recognize that bi-functional shRNA design targeting mesothelin provides an advantageous formulation to knockdown the expression of MSLN for the treatment of cancer.

Mesothelin (MSLN) is found with a glycosylphosphatidylinositol linkage on the surface of, e.g., human pancreatic ductal adenocarcinomas (PDAC). Overexpressed MSLN may function as a mediator of cell survival under anchorage-independent conditions, where it facilitates anchorage-independent growth and confers resistance to anoikis. The present inventors recognize that overexpression of MSLN in pancreatic cancer cells leads to increased proliferation and survival as well as an increased invasive phenotype in vitro with increased tumor growth and metastasis in vivo. The present inventors recognize that the importance of MSLN in PDAC tumorigenesis, along with its limited expression in normal tissues, makes it an attractive therapeutic target. To silence the expression of MSLN, bi-functional shRNA technology is employed, which demonstrates a more effective and durable silencing of the target gene expression by concurrently inducing translational repression through mRNA sequestration in p bodies as well as post-transcriptional mRNA cleavage. Two plasmid vectors expressing bi-functional shRNA targeting MSLN were designed and tested, with the objective of determining the effects of these novel vectors on pancreatic cancer cell proliferation and migration in vitro through regulation of MSLN.

In certain embodiments, the invention includes bi-functional shRNA design and formulations to provide improved RNA interference formulation for enhanced efficacy targeting MSLN for the treatment of insulinoma, pancreatic and related cancer.

RNAi is the Nobel prize winning discovery by Fire and Mello in 1998, which has fostered an exponential number of studies and publications furthering the understanding of gene function and stimulating numerous phase I and II clinical trials. This naturally occurring gene-silencing mechanism by small RNAs, which includes endogenous microRNA (miRNA), is highly dependent on gene sequence; thus the mechanism can, in theory, be used to inhibit the expression of any targeted gene[s] with strong specificity. RNAi is not limited by the pharmacologic constraints inherent to the development of small molecules which creates an opportunity to access traditionally “undruggable” targets for disease treatment.

The central player of this mechanism is the RNA Induced Silencing Complex (RISC). The process starts with double-stranded small RNA (composed of a passenger strand and a guide strand) which is incorporated into the pre-RISC followed by the cleavage—dependent or cleavage—independent release of the passenger strand to form the guide strand containing RISC. The guide strand (anti-sense to mRNA) guides the RISC to recognize the target mRNA through sequence complementarity (full or extended partial). A key component of RISC is the family of Argonaute proteins (Ago), Ago 1, 2, 3 and 4 in mammalian systems, of which only Ago 2 has endonuclease activity so as to allow for cleavage of the target mRNA for further degradation (cleavage dependent pathway); all the Ago containing RISC can function through a cleavage-independent effector pathway resulting in translation repression and mRNA sequestration in p-body with subsequent degradation. The cleavage-dependent effector process requires extensive homology between guide strand and both the passenger strand and target mRNA, particularly in the central region; the cleavage-independent effector process, on the other hand, only requires partial homology between guide strand and both the passenger strand and target mRNA.

The present invention takes advantage of both cleavage dependent and cleavage independent loading at the RISC complex, not the events that are downstream from the RISC complex. Thus, as used herein the phrase “cleavage dependent and cleavage independent” refers to the design of RNA(s) that are specifically targeted to RISC and the cleavage dependent and cleavage independent activities at the RISC complex, i.e., loading. It has been found herein and in the parent application for this case, that these “bifunctional shRNAs” have a higher inhibitory activity than the sum of targeting each individual part of the RISC complex. Thus, the higher inhibitory activity of the present invention.

RNA interference can be triggered either by synthetic double stranded small interfering RNA (siRNA) or by vector driven short hairpin RNA (shRNA). Both siRNA and vector driven shRNA have been demonstrated to be effective in in vitro and in vivo applications, each with their respective advantages. Most siRNA are structurally designed to promote efficient incorporation into the Ago2 containing RISC, the RNase III containing Dicer-substrate design improves the efficiency of siRNA at least 10-fold by initial association and processing at the pre-RISC. Vector driven shRNA utilizes the host microRNA biogenesis pathway, which appears to be more efficient. siRNA is more readily chemically modified while shRNA expression can be modulated and regulated by specific promoters.

The present inventors developed the novel vector driven shRNA technology, the bi-functional shRNA (bi-shRNA), to further improve the efficiency of RNAi by harnessing both cleavage-dependent and cleavage-independent pathways of RISC loading in one pre-programmed molecule. The vector driven bi-shRNA includes two stem-loop structures for each mRNA target sequence, one stem-loop shRNA has perfect complementarity at the stem and the second stem-loop shRNA contains mismatches on the passenger strand of the stem (thereby differing from prior art mismatched RNA that include the mismatch on the guide strand). Importantly, following incorporation into the RISC, the guide strands derived from each of the two structures are fully complementary to the mRNA target sequence but are associated with different Ago containing RISCs. The bi-shRNA design leads to more rapid onset of gene silencing, higher efficacy, and greater durability when compared with either siRNA or conventional shRNA. Currently personalized cancer therapy with target specific bi-shRNA is transitioned into the clinic in Phase I studies using a modified bilamellar invaginated liposome delivery vehicle. Key molecular methods involved in design, construction, and the implementation of bi-shRNA are provided below.

Depending on that objective and the embodiments, several different vectors, promoters, or plasmid backbones and delivery systems can be used. It has been found useful to choose an expression vector with efficient transgene expression. The present inventors recognized that an expression vector with powerful promoters, e.g., an extended CMV promoter containing IE 5′UTR and partial Intron A (pUMVC3), is more effective than those with a cloning site immediately adjacent to the CMV promoter. In certain embodiments it is beneficial to have a stretch of lead transcript before the stem-loop structures. In addition, if more than one vector usage is planned, an effective shuttle strategy should be worked out beforehand; modification by PCR amplification of the expressed cassette is not as efficient. The choice of promoter is also important; RNA polymerase III promoters are much stronger in expression but competitively saturate the endogenous miRNA maturation process at both the nuclear export and RISC loading steps resulting in lethal toxicity in vitro and in vivo with certain delivery vehicles. RNA polymerase II promoters, although less strong in expression, works efficiently and is much less toxic vis-à-vis competition for the endogenous miRNA pathway.

In certain embodiments a sequence that can act in more than one species is designed, particularly if multiple animal model systems are utilized. For most target genes, it is possible to find stretches of target nucleotides that are conserved between species. For finding a sequence that is both conserved and optimum for knockdown, one has to compare the homology-matched sequence with the selected target site sequence.

Public accessible computer programs using differing algorithms (e.g. Dharmacon RNAi design center (www.dharmacon.com) and IDT (www.idtdna.com) are readily available and can be used to locate appropriate target sites within the targeted gene. A search with most computer programs will often yield a preliminary first set of targets for further analysis. Some available publications offer do and do-not suggestions. A BLAST search for each target sequence is to be taken in order to analyze potential cross homology with other mRNAs within the species of interest.

Once the target site sequence is selected, the bi-shRNA design process can begin; the design process is presented below. The bi-shRNA stem-loop structure used by the inventors employs the well-analyzed miR-30a backbone, although, any functional miRNA backbone can be used. The bi-shRNA consists of the two stem-loop structures on a miR-30a backbone located immediately adjacent to each other with a gap about 10 nucleotides long. A longer nucleotide gap can be used and multiple units of bi-sh RNA can be designed to string together in a single transcript targeting either a single gene at multiple sites or multiple different genes simultaneously.

To construct the expression unit to be placed in the multiple cloning sites of an expression vector, an assembly strategy using synthetic oligonucleotides sequentially linked together has been developed. Alternatively, one can also outsource the synthesis of the gene construct with the specified sequence to a biotechnology service company. For the oligonucleotide assembly process, overlapping DNA fragments were designed and synthesized. Because of redundant sequences in the two stem-loop structures, it is necessary to initially ligate the 5′ fragments and 3′ fragments. The 5′ fragment and the 3′ fragment can then be purified on gel and further ligated to the middle linking fragments. This assembly process is efficient and, with careful design, many fragments can be repetitively used for different bi-functional constructs.

For each target, it is the best to design and construct at least three bi-functional constructs to compare and from which to select a construct with high knockdown efficiency for further evaluation. Knockdown efficiency can be compared in vitro in tissue culture cells. The present inventors recognized that is generally difficult to compare the knockdown efficiency with endogenously expressed genes because in vitro transfection methods have widely different efficiencies; this is particularly so when the transfection efficiency is low as the knockdown is hard to assess due to background noise from untransfected cells.

Efficacy and efficiency of target gene knockdown by bi-shRNA can be tested with a variety of in vitro and in vivo systems depending on the target and planned application. This in vitro assessment can be conducted following transfection of the bi-shRNA expression plasmids in a variety of cultured cells. The present inventors found that transfections by both electroporation and by liposome (e.g., Lipofectamine 2000) are highly effective, when the amount of plasmid DNA is carefully controlled using a control vector or universal random sequence. For Lipofectamine or a related agent, the present inventors found that the reverse transfection method, in general, is less toxic than the forward transfection method. Target gene knockdown can be assessed by either qRT-PCR for target gene mRNA or by Western and/or ELISA for target gene protein. In one assay methods the expression of mature shRNA by stem-loop RT-PCR is detected, in another essay method, the target mRNA cleavage is detected by 5′ RNA-Ligand Mediated RACE (5′ RLM-RACE). Stem-loop RT-PCR is a sensitive method dependent on the specific probe primer used; in addition, one can specifically detect and quantify both the passenger strand and guide strand. For bi-shRNA, the method can differentially score both the fully complementary as well as the mismatched (partially complementary) passenger strand. The 5′ RLM-RACE method requires ligation of an RNA oligomer onto the cleaved mRNA end, consequently, the method is rendered less efficient. Insofar as a number of rounds of amplifications are often required, a nested primer design is essential to ensure specificity.

Evaluable functionality of bi-shRNA relies on effective plasmid delivery into target cells. The inventors recognize that some in vitro transfection systems often do not translate to inherently more complex in vivo animal models. There are numerous delivery systems designed specifically for systemic applications in vivo. The present inventors utilize the fusogenic, cationic DOTAP:cholesterol bilamellar invaginated vesicle lipoplex (BIV) for in vivo studies and has successfully translated it to the clinic. Modification strategies for more focused biodistribution, targeted delivery, and enhanced intracellular uptake are developed. An effective lipoplex should use plasmids devoid of any contaminants from host E. coli. Although endo-free plasmid purification kit produced plasmids are generally used, GLP or GMP produced plasmids are more effective. Unfortunately, colanic acid and other non-endotoxin associated polysaccharides co-purify with DNA by anion exchange chromatography and by cesium chloride density gradient centrifugation. Therefore, endotoxin removal does not remove these contaminants, and HPLC cannot detect these contaminants. To correct this, the Superclean™ procedure has been developed to generate ultra-high quality plasmid DNA, cleansed of these contaminants, for in vivo and clinical applications. Liposome preparation involves highly specialized equipment; the present inventors routinely generate the DOTAP:cholesterol BIV in a GMP facility. Pre-made liposome may be obtained from a collaborator or purchased from a vendor. The process of preparing lipoplex with high quality liposome and plasmid DNA is described below. The lipoplex formulation can be achieved in most laboratory settings. Once the lipoplex is made, the formulation can be delivered systemically to experimental animals either through slow tail vein injection or with catheters. Target site vector expression can be analyzed using the PCR method for plasmid DNA and the stem-loop RT-PCR for mature bi-shRNA, respectively. bi-shRNA functionality can be assayed with the 5′ RLM-RACE for target mRNA cleavage and with Western blot or IHC for target protein knockdown. These analyses can be performed at about 48 hours post treatment. For efficacy, repeated delivery into the experimental animal is often required; the dosing schedule needs to be experimentally determined and optimized.

The invention provides that target gene-specific shRNAs may be designed to enter into and interact with the cleavage-dependent RISC and cleavage-independent RISC pathways. As used herein, the term “bifunctional shRNA” generally means one or more RNA molecules, each of which include a double stranded sequence that resides within a stem portion of separate stem-loop structures, wherein a first RNA molecule is designed to be presented to a cleavage-dependent RISC pathway and a second RNA molecule is designed to be presented to a cleavage-independent RISC pathway. In certain embodiments, the bi-shRNA is all on a single strand.

More specifically, a first guide strand sequence is complementary, preferably 100% complementary, to at least a portion of an mRNA transcript encoded by a target gene. The invention provides that this guide strand (which is initially bonded to the passenger strand to form the double stranded stem) comprises a nucleic acid sequence that is capable of binding to the mRNA transcript of the target gene, and is presented to the cleavage-dependent RISC pathway. The invention provides that such binding of the guide strand sequence to the mRNA transcript, and presentation to the cleavage-dependent RISC pathway, causes degradation of the mRNA transcript.

In particular embodiments, it is provided that the second guide strand sequence is at least partially complementary to at least a portion of the mRNA transcript encoded by the target gene. More particularly, the second guide strand sequence may contain a first portion that is complementary, preferably 100% complementary, to the mRNA transcript encoded by the target gene, whereas a second portion of the guide strand sequence contains certain bases that are mismatched with the corresponding sequence of the target gene mRNA transcript.

As used herein, a “mismatched” base pair refers to two nitrogenous bases within a nucleic acid sequence that, when bound (or hybridized) to each other, do not follow Chargaffs rules of base pairing. Chargaffs rules provide that the purine adenine (A) within a first nucleic acid sequence will pair with the pyrimidine thymine (T) (or uridine (U)) within a second nucleic acid sequence. Furthermore, Chargaffs rules provide that the purine guanine (G) within a first nucleic acid sequence will pair with the pyrimidine cytosine (C) within a second nucleic acid sequence. Thus, a base pairing between two strands (nucleic acid sequences) that does not follow and comply with such rules would be deemed a “mismatched” base pair, e.g., a pairing between G and U, A and G, A and C, G and T, G and U, and so on. A guide strand within the double stranded sequence of the stem-loop structures shown therein, which contain one or more “mismatched” base pairs relative to the passenger strand, creates a bulge in the double stranded stem sequence.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., a-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “expression vector” as used herein in the specification and the claims includes nucleic acid molecules encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter. The term “promoter” refers to any DNA sequence which, when associated with a structural gene in a host yeast cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

The term “oncogene” as used herein refers to genes that permit the formation and survival of malignant neoplastic cells (Bradshaw, T. K.: Mutagenesis 1, 91-97 (1986).

As used herein the term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

The term “hybridizing” refers to any process by which a strand of nucleic acid binds with a complementary strand through base pairing.

The term “transfection” refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein the term “bi-functional” refers to a shRNA having two mechanistic pathways of action, that of the siRNA and that of the miRNA. The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein, the term “liposome” refers to a closed structure composed of lipid bilayers surrounding an internal aqueous space. The term “polycation” as used herein denotes a material having multiple cationic moieties, such as quaternary ammonium radicals, in the same molecule and includes the free bases as well as the pharmaceutically-acceptable salts thereof.

Accordingly, the bifunctional shRNAs may comprise shRNAs designed to enter into and interact with both cleavage-dependent RISC and cleavage-independent RISC. A higher level of gene “knock-down” is achieved using such bifunctional shRNAs compared to other currently-available RNAi methods and compositions, including siRNAs and conventional shRNAs (i.e., shRNA constructs designed to enter cleavage-dependent RISC or cleavage-independent RISC, but not both).

As used herein, gene “knock-down” refers to effective quantitative and durable inhibition of expression. Such gene “knock-down” may be manifested, and/or apparent, in the suppression of target gene mRNA translation, increased target cell apoptosis and/or cell kill.

As used herein, “target gene” refers to a nucleic acid sequence in a cell, wherein the expression of the sequence may be specifically and effectively modulated using the bifunctional shRNA. In certain embodiments, the target gene may be implicated in the growth (proliferation), maintenance (survival), and/or migratory (metastatic) behavior of an individual's cancer. The invention provides, however, that the target gene may be implicated in any other disease or medical condition, and is not limited to genes implicated in cancer. For example, the target gene may represent any sequence that an investigator or clinician wishes to silence (i.e., reduce the expression level of such target gene).

Vector sequence may comprise a promoter, which is operably linked (or connected), directly or indirectly, to a sequence encoding the bifunctional shRNAs. Such promoters may be selected based on the host cell and the effect sought. Non-limiting examples of suitable promoters include constitutive and inducible promoters, such as inducible RNA polymerase II (pol II)-based promoters. Non-limiting examples of suitable promoters further include the tetracycline inducible or repressible promoter, RNA polymerase I or III-based promoters, the pol II dependent viral promoters, such as the CMV-IE promoter, and the pol III U6 and H1 promoters. The bacteriophage T7 promoter may also be used (in which case it will be appreciated that the T7 polymerase must also be present). The invention shall not be restricted to the use of any single promoter, especially since the invention may comprise two or more bifunctional-shRNAs (i.e., a combination of effectors), including but not limited to incorporated shRNA singlets. Each incorporated promoter may control one, or any combination of, the shRNA singlet components.

In certain embodiments, the promoter may be preferentially active in the targeted cells, e.g., it may be desirable to preferentially express the bifunctional shRNA molecules in tumor cells using a tumor cell-specific promoter. Introduction of such constructs into host cells may be effected under conditions whereby the two or more RNA molecules that are contained within the bifunctional shRNA precursor transcript initially reside within a single primary transcript, such that the separate RNA molecules (each comprising its own stem-loop structure) are subsequently excised from such precursor transcript by an endogenous ribonuclease. The invention further provides that splice donor and acceptor sequences may be strategically placed within the primary transcript sequence to promote splice some-mediated nuclear processing. The resulting mature shRNAs may then induce degradation, and/or translation repression, of target gene mRNA transcripts produced in the cell. Alternatively, each precursor stem-loop structure may be produced as part of a separate transcript, in which case each shRNA-encoding sequence will preferably include its own promoter and transcription terminator sequences. Additionally, the bifunctional shRNA precursor transcript may reside within a single primary transcript, which, optionally, further comprises of one or more mRNA sequences that encode one or more functional mammalian proteins. For example, the one or more mRNA sequences may encode certain proteins that are known to bolster a patient's immune system, or otherwise provide some preventative and/or therapeutic effect that will operate in parallel with the bifunctional shRNA.

The stem-loop structures of the shRNA molecules described herein may be about 40 to 100 nucleotides long or, preferably, about 50 to 75 nucleotides long. The stem region may be about 19-45 nucleotides in length (or more), or more preferably about 20-30 nucleotides in length. The stem may comprise a perfectly complementary duplex (but for any 3′ tail), however, bulges or interior loops may be present, and even preferred, on either arm of the stem. The number of such bulges and asymmetric interior loops are preferably few in number (e.g., 1, 2 or 3) and are about 3 nucleotides or less in size. The terminal loop portion may comprise about 4 or more nucleotides, but preferably not more than about 25. More particularly, the loop portion will preferably be 6-15 nucleotides in size.

As described herein, the stem regions of the bifunctional shRNAs comprise passenger strands and guide strands, whereby the guide strands contain sequences complementary to the target mRNA transcript encoded by the target gene(s). Preferably, the G-C content and matching of guide strand and passenger strand is carefully designed for thermodynamically-favorable strand unwind activity with or without endonuclease cleavage. Furthermore, the specificity of the guide strand is preferably confirmed via a BLAST search (www.ncbi.nim.nih.qov/BLAST).

Expression level of multiple target genes may be modulated using the methods and bifunctional shRNAs described herein. For example, the invention provides that a first set of bifunctional shRNAs may be designed to include a sequence (a guide strand) that is designed to reduce the expression level of a first target gene, whereas a second set of bifunctional shRNAs may be designed to include a sequence (a guide strand) that is designed to reduce the expression level of a second target gene. The different sets of bifunctional shRNAs may be expressed and reside within the same, or separate, preliminary transcripts. In certain embodiments, such multiplex approach, i.e., the use of the bifunctional shRNAs described herein to modulate the expression level of two or more target genes, may have an enhanced therapeutic effect on a patient. For example, if a patient is provided with the bifunctional shRNAs described herein to treat, prevent, or ameliorate the effects of cancer, it may be desirable to provide the patient with two or more types of bifunctional shRNAs, which are designed to reduce the expression level of multiple genes that are implicated in the patient's cancer.

In certain embodiments, the invention further provides that the bifunctional shRNA sequences may comprise stem sequences of naturally occurring miRNAs (e.g., miR-30, C. elegans let-7 and/or lin-4). While the presence of a miR-30 loop, for example, may be desirable, the invention provides that variations of that structure may be tolerated, wherein loops may be used that are greater than 72%, preferably greater than 79%, more preferably greater than 86%, and most preferably, greater than 93% identical to, for example, the miR-30 sequence (determined using well-known computer programs such as the BESTFIT program (Wisconsin Sequence Analysis Package, Version 8 for Unix, Genetics Computer Group, University Research Park, 575 Science Drive, Madison, Wis. 53711)).

The precursor sequences (or constructs) encoding the bifunctional shRNAs may be introduced into host cells using any of a variety of techniques and delivery vehicles well-known in the art. For example, infection with a viral vector comprising one or more constructs may be carried out, wherein such viral vectors preferably include replication defective retroviral vectors, adenoviral vectors, adeno-associated vectors, lentiviral vectors, or measle vectors. In addition, transfection with a plasmid comprising one or more constructs may be employed. Such plasmids may be present as naked DNA, or may be present in association with, for example, a liposome (e.g., an immunoliposome). Still further, the delivery vehicle may consist of immunolipoplexes, targeted nanoparticles, targeted liposomes, cyclodextrins, nanoparticles, aptamers, dendrimers, chitosan, or pegylated derivatives thereof. The nature of the delivery vehicle may vary depending on the target host cell.

In-vivo delivery of the bifunctional shRNA-encoding constructs may be carried out using any one of a variety of techniques, depending on the target tissue. Delivery may be, for example, achieved by direct injection, inhalation, intravenous injection or other physical methods (including via micro-projectiles to target visible and accessible regions of tissue (e.g., with naked DNA). Administration may further be achieved via syringe needles, trocars, canulas, catheters, etc., as appropriate.

In addition to the methods of using the bifunctional shRNAs described herein, provided for are shRNAs themselves. Accordingly, additional aspects include nucleic acid sequences, which may comprise a single contiguous sequence or multiple distinct sequences that, individually or collectively, encode two or more RNA molecules. According to such embodiments, a first RNA molecule will comprise a double stranded sequence that includes a guide strand sequence that is complementary to a portion of an mRNA transcript encoded by a target gene, whereas a second RNA molecule comprises a second double stranded sequence that includes a second guide strand sequence that is partially complementary to a portion of such mRNA transcript. Preferably, the second guide strand sequence of the second RNA molecule comprises one or more bases that are mismatched with a nucleic acid sequence of the mRNA transcript encoded by the target gene. According to further aspects, expression vectors are provided which comprise the nucleic acid sequences, and may be used to carry out the methods, and express the bifunctional shRNAs, described herein.

Still further, methods of using the nucleic acid sequences and bifunctional shRNAs are described herein to prevent, treat and/or ameliorate the effects of one or more medical conditions, including without limitation various types of cancer. For example, the invention provides that the bifunctional shRNAs described herein may be used to reduce the expression level of one or more target genes that are implicated in cancer cell growth, survival, and/or metastasis. For example, as demonstrated in the Examples below, the bifunctional shRNAs may be used to reduce the expression level of certain target genes that encode scaffold proteins, which have been found to be over-expressed in cancer cells. Non-limiting examples of such target genes include Mesothelin.

RNA Interference: The introduction of artificial double-stranded small interfering RNAs (siRNAs) into animal and plant cells can induce the degradation of targeted mRNA molecules with complementary sequence; the process is known as RNA interference (RNAi) (Sharp 2001; Hutvagner and Zamore 2002; Zamore 2002) (see U.S. Pat. No. 6,506,559). RNAi has emerged as a useful experimental tool with strong potential for therapeutic applications (Fire, Xu et al. 1998; Hammond, Bernstein et al. 2000; Elbashir, Harborth et al. 2001; Senzer, Rao et al. 2009; Wang Z 2011). However, in mammalian cells, induction of RNAi using shRNAs requires the transfection of RNA oligonucleotides, which can be inefficient with the duration of effective silencing limited by vehicle disassembly time and siRNA biologic half life. Despite these limitations, in a recent early results publication of a clinical phase I study, Davis and colleagues have convincingly demonstrated target specific silencing and site-specific cleavage with systemic delivery of a pegylated, transferrin decorated, cyclodextrin-based siRNA targeting the M2 subunit of ribonucleotide reductase (RRM2) (CALAA-01) (Davis, Zuckerman et al. 2010). Three reported patients with biopsy accessible melanoma, who were treated as per the dose-escalation Phase I study, received 18, 24, or 30 mg/m2 CALAA-01 by intravenous infusion on days 1, 3, 8, and 10 of a 21 day cycle. Voluntary biopsies were performed after the final dose of cycle 1 in each and compared to archived tumor, and at 1 month post cycle 1 (prior to initiation of cycle 2) and on the day of the final dose of cycle 2 in the patient treated at 30 mg/m2. RRM2 mRNA reduction was confirmed by qRT-PCR comparing post- and pre-cycle 2 tissue samples at 30 mg/m2. In the same patient, immunohistochemistry and Western blot pre- and post-cycle 1 showed a five-fold reduction in MMR2 protein. Supporting the proposed mechanism of action, 5′-RLM-RACE (5′-RNA-ligase-mediated rapid amplification of complementary DNA ends) confirmed the predicted cleavage site. This first-in-human demonstration of targeted tumor cell entry (using transmission electron microscopy) and mRNA and protein expression reduction along with predicted site-specific siRNA cleavage following systemic delivery brings added impetus to translational application of RNAi.

siRNA requires chemical modification to increase serum stability, cellular uptake and duration of action. Alternatively, siRNA can be constructed as a short hairpin RNA (shRNA). shRNA consists of a stem-loop structure that can be transcribed in cells from RNA polymerase III (or, less frequently used, RNA polymerase II) promoter on a plasmid construct (Miyagishi and Taira 2002; Yu, DeRuiter et al. 2002). Constitutive expression of shRNA from a plasmid independently from the chromosome provides an advantage over synthetic siRNA. The shRNA expression units can be incorporated into a variety of plasmids and viral vectors for intracellular delivery and nuclear import. In addition, vector based shRNA expression can also be regulated or induced (Gossen and Bujard 1992; Gupta, Schoer et al. 2004; Dickins, Hemann et al. 2005). shRNAs, as opposed to synthetic siRNAs, are synthesized in the nucleus of cells, then processed and transported to the cytoplasm to be incorporated into the RNA-induced silencing complex (RISC) for activity (Cullen 2005). To be effective, shRNA has to be designed to utilize the endogenous cellular microRNA biogenesis machinery.

Bifunctional shRNA: As described above, RNA interference (RNAi) is a natural cellular regulatory process capable of inhibiting transcriptional, post-transcriptional and translational mechanisms thereby modulating gene expression. Using a miR30-scaffold, the present inventors developed a “bifunctional” RNAi strategy which demonstrated more effective silencing of target gene expression by concurrently inducing translational repression, and [GW 182-mediated] sequestration in the p-body (as a holding reservoir or promoting de-capping, de-adenylation and mRNA degradation) (cleavage-independent) as well as post-transcriptional mRNA mRNA cleavage (cleavage dependent) (Rao D 2010).

Expression of MSLN in high endogenous MSLN expressing pancreatic cancer cells ASPC1 as well as in MIA-PaCa2 cells with forced MSLN expression (MIA-MSLN) was determined by quantitative reverse transcription PCR (qRT-PCR) and western blotting before and after transfection with bifunctional vectors or a control vector pUMVC3 for 48 hours. The levels of MSLN-modulated downstream targets, such as STAT3 expression and NF-κB induction, were measured to confirm the functional consequence of MSLN down-regulation. In vitro cell proliferation and migration were determined by a MTT assay and a modified Boyden chamber assay, respectively. As a result, MSLN expression was reduced significantly in both ASPC1 and MIA-MSLN cell lines following transfection of bi-functional vectors, as confirmed at both the mRNA and protein levels. Down-regulation of MSLN led to a decrease in the levels of MSLN-responsive tumorigenic factors, including STAT3 and NF-kB, and a concomitant significant decrease in both proliferation and migration of tumor cells.

Bi-functional shRNA vectors targeting MSLN result in a significant down-regulation in MSLN expression at both the mRNA and protein levels. Down-regulation of MSLN effectively reduces STAT3 and NF-kB induction, resulting in inhibition of cell proliferation and cell migration in human pancreatic cancer cell lines that highly express MSLN. These data show that bi-functional shRNA vectors are a novel and specific therapeutic agent for the treatment of MSLN-overexpressing human pancreatic cancers, and can be employed in cancer treatment.

MSLN expression knockdown was also demonstrated with pancreatic cancer cell line Capan-2. FIG. 6 shows the expression of MSLN in MIA-MSLN (forced MSLN expression stable cell line), Capan-2 (endogenous MSLN high expression cell line), and MIA-V (expression vector control stable cell line) by using western blot. As shown below, MIA-V has very low MSLN expression while both MIA-MSLN and Capan-2 cells have high MSLN expression.

Capan-2 cell or MIA-MSLN cells transfected with GFP expression vectors, FIG. 7 shows more than 90% of cells were efficiently transfected 24 hours post transfection. When MIA-MSLN and Capan-2 cells were transfected with sh33 (bi-shRNA against MSLN), sh02 (positive control MSLN knock-down plasmid obtained from a commercial source), or pUMVC3 (vector control) and MSLN mRNA and protein expression levels were monitored at 72 hours post-transfection, sh33 preforms better than conventional sh02.

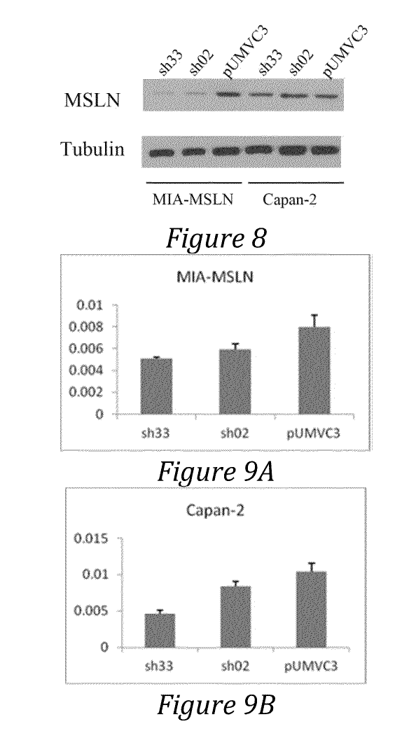

FIG. 9 shows that at 72 h post transfection, RNA and protein were extracted from the transfected cells. A qRT-PCR was run to compare the MSLN mRNA expression level changes upon different plasmids transfection in both cell lines. As shown in the qRT-PCR, both sh33 and sh02 can significantly reduce MSLN mRNA level to about 50% when compared with control pUMVC3 in both MIA-MSLN cells (FIG. 9a) and Capan-2 cells (FIG. 9b), with sh33 showed a better knock-down effect than sh02 in silencing MSLN at the mRNA level. Sh33 preforms much better than sh02 at mRNA knockdown in Capan-2 cells.

FIG. 8 shows both sh33 and sh02 are able to reduce MSLN protein expression and that sh33 has a comparable MSLN knock-down effect to sh02 in MIA-MSLN cells. However, for the Capan-2, neither sh33 nor sh02 showed significant protein expression knockdown. This experiment has been repeated several times with similar results. The observation that the MSLN mRNA can be effectively reduced by sh33 while MSLN protein was not significantly reduced is not expected. One explanation is the MSLN protein in Capan-2 is more stable with much slower turnover rate so that the total MSLN protein was not significantly reduced within the time monitored. We are continuing our study to monitor both mRNA and protein knockdown with a broader time course. We are also preparing stably transformed Capan-2 cell lines to study the knockdown effect on downstream signaling targets and biological effects of knockdown.

In one embodiment, MSLN target sites on mRNA are located within the Homo sapiens mesothelin (MSLN), transcript variant 1, mRNA (gi|68303642|ref|NM_{—}005823.4):

(SEQ ID NO: 1)
AGAGCTACCGGTGGACCCACGGTGCCTCCCTCCCTGGGATCTACACAGA
CCATGGCCTTGCCAACGGCTCGACCCCTGTTGGGGTCCTGTGGGACCCC
CGCCCTCGGCAGCCTCCTGTTCCTGCTCTTCAGCCTCGGATGGGTGCAG
CCCTCGAGGACCCTGGCTGGAGAGACAGGGCAGGAGGCTGCGCCCCTGG
ACGGAGTCCTGGCCAACCCACCTAACATTTCCAGCCTCTCCCCTCGCCA
ACTCCTTGGCTTCCCGTGTGCGGAGGTGTCCGGCCTGAGCACGGAGCGT
GTCCGGGAGCTGGCTGTGGCCTTGGCACAGAAGAATGTCAAGCTCTCAA
CAGAGCAGCTGCGCTGTCTGGCTCACCGGCTCTCTGAGCCCCCCGAGGA
CCTGGACGCCCTCCCATTGGACCTGCTGCTATTCCTCAACCCAGATGCG
TTCTCGGGGCCCCAGGCCTGCACCCGTTTCTTCTCCCGCATCACGAAGG
CCAATGTGGACCTGCTCCCGAGGGGGGCTCCCGAGCGACAGCGGCTGCT
GCCTGCGGCTCTGGCCTGCTGGGGTGTGCGGGGGTCTCTGCTGAGCGAG
GCTGATGTGCGGGCTCTGGGAGGCCTGGCTTGCGACCTGCCTGGGCGCT
TTGTGGCCGAGTCGGCCGAAGTGCTGCTACCCCGGCTGGTGAGCTGCCC
GGGACCCCTGGACCAGGACCAGCAGGAGGCAGCCAGGGCGGCTCTGCAG
GGCGGGGGACCCCCCTACGGCCCCCCGTCGACATGGTCTGTCTCCACGA
TGGACGCTCTGCGGGGCCTGCTGCCCGTGCTGGGCCAGCCCATCATCCG
CAGCATCCCGCAGGGCATCGTGGCCGCGTGGCGGCAACGCTCCTCTCGG
GACCCATCCTGGCGGCAGCCTGAACGGACCATCCTCCGGCCGCGGTTCC
GGCGGGAAGTGGAGAAGACAGCCTGTCCTTCAGGCAAGAAGGCCCGCGA
GATAGACGAGAGCCTCATCTTCTACAAGAAGTGGGAGCTGGAAGCCTGC
GTGGATGCGGCCCTGCTGGCCACCCAGATGGACCGCGTGAACGCCATCC
CCTTCACCTACGAGCAGCTGGACGTCCTAAAGCATAAACTGGATGAGCT
CTACCCACAAGGTTACCCCGAGTCTGTGATCCAGCACCTGGGCTACCTC
TTCCTCAAGATGAGCCCTGAGGACATTCGCAAGTGGAATGTGACGTCCC
TGGAGACCCTGAAGGCTTTGCTTGAAGTCAACAAAGGGCACGAAATGAG
TCCTCAGGTGGCCACCCTGATCGACCGCTTTGTGAAGGGAAGGGGCCAG
CTAGACAAAGACACCCTAGACACCCTGACCGCCTTCTACCCTGGGTACC
TGTGCTCCCTCAGCCCCGAGGAGCTGAGCTCCGTGCCCCCCAGCAGCAT
CTGGGCGGTCAGGCCCCAGGACCTGGACACGTGTGACCCAAGGCAGCTG
GACGTCCTCTATCCCAAGGCCCGCCTTGCTTTCCAGAACATGAACGGGT
CCGAATACTTCGTGAAGATCCAGTCCTTCCTGGGTGGGGCCCCCACGGA
GGATTTGAAGGCGCTCAGTCAGCAGAATGTGAGCATGGACTTGGCCACG
TTCATGAAGCTGCGGACGGATGCGGTGCTGCCGTTGACTGTGGCTGAGG
TGCAGAAACTTCTGGGACCCCACGTGGAGGGCCTGAAGGCGGAGGAGCG
GCACCGCCCGGTGCGGGACTGGATCCTACGGCAGCGGCAGGACGACCTG
GACACGCTGGGGCTGGGGCTACAGGGCGGCATCCCCAACGGCTACCTGG
TCCTAGACCTCAGCATGCAAGAGGCCCTCTCGGGGACGCCCTGCCTCCT
AGGACCTGGACCTGTTCTCACCGTCCTGGCACTGCTCCTAGCCTCCACC
CTGGCCTGAGGGCCCCACTCCCTTGCTGGCCCCAGCCCTGCTGGGGATC
CCCGCCTGGCCAGGAGCAGGCACGGGTGGTCCCCGTTCCACCCCAAGAG
AACTCGCGCTCAGTAAACGGGAACATGCCCCCTGCAGACACGT
Bold area = coding sequences (SEQ ID NO: 2)
Underlined area 1 = target site 1: GCCTCATCTTCTAC
AAGAA (SEQ ID NO: 3)
Underlined area 2 = target site 2: CCAGGACCAGCAGG
AGGCA (SEQ ID NO: 4)

In certain embodiments, bi-shRNA-MSLN-1 comprises the following sequences:

(SEQ ID NO: 3)
Sense sequence: 5′ GCCTCATCTTCTACAAGAA
(SEQ ID NO: 5)
Antisense sequence: 5′TTCTTGTAGAAGATGAGGC

The insert for bi-shRNA-MSLN-1 vector (pGBI-33) is the following:

(SEQ ID NO: 6)
TCGACTGCTGTTGAAGTGAGCGCCGCCTCATCTTCTACAAGAATAGTGAAGCCA
CAGATGTATTCTTGTAGAAGATGAGGCGTTGCCTACTGCCTCGGAAGCAGCTCACTACA
TTACTCAGCTGTTGAAGTGAGCGCCGCCTCATCGAATACAAGAATAGTGAAGCCACAG
ATGTATTCTTGTAGAAGATGAGGCGTTGCCTACTGCCTCGGAAGCTTAATAAAGGATCT
TTTATTTTCATTGGC

The full sequence for pGBI-33 (bi-shRNA-MSLN-1) is the following:

(SEQ ID NO: 9)
TGGCCATTGCATACGTTGTATCCATATCATAATATGTACATTTATATTGGCTCAT
GTCCAACATTACCGCCATGTTGACATTGATTATTGACTAGTTATTAATAGTAATCAATT
ACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATG
TTCCCATAGTAACGCCAATAGGGACTTTCCATTGACGTCAATGGGTGGAGTATTTACGG
TAAACTGCCCACTTGGCAGTACATCAAGTGTATCATATGCCAAGTACGCCCCCTATTGA
CGTCAATGACGGTAAATGGCCCGCCTGGCATTATGCCCAGTACATGACCTTATGGGACT
TTCCTACTTGGCAGTACATCTACGTATTAGTCATCGCTATTACCATGGTGATGCGGTTTT
GGCAGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGT
CTATATAAGCAGAGCTCGTTTAGTGAACCGTCAGATCGCCTGGAGACGCCATCCACGCT
GTTTTGACCTCCATAGAAGACACCGGGACCGATCCAGCCTCCGCGGCCGGGAACGGTG
CATTGGAACGCGGATTCCCCGTGCCAAGAGTGACGTAAGTACCGCCTATAGACTCTAT
AGGCACACCCCTTTGGCTCTTATGCATGCTATACTGTTTTTGGCTTGGGGCCTATACACC
CCCGCTTCCTTATGCTATAGGTGATGGTATAGCTTAGCCTATAGGTGTGGGTTATTGAC
CATTATTGACCACTCCAACGGTGGAGGGCAGTGTAGTCTGAGCAGTACTCGTTGCTGCC
GCGCGCGCCACCAGACATAATAGCTGACAGACTAACAGACTGTTCCTTTCCATGGGTCT
TTTCTGCAGTCACCGTCGTCGACTGCTGTTGAAGTGAGCGCCGCCTCATCTTCTACAAG
AATAGTGAAGCCACAGATGTATTCTTGTAGAAGATGAGGCGTTGCCTACTGCCTCGGA
AGCAGCTCACTACATTACTCAGCTGTTGAAGTGAGCGCCGCCTCATCGAATACAAGAA
TAGTGAAGCCACAGATGTATTCTTGTAGAAGATGAGGCGTTGCCTACTGCCTCGGAAG
CTTAATAAAGGATCTTTTATTTTCATTGGCGGCCGCGGATCCAGATCTTTTTCCCTCTGC
CAAAAATTATGGGGACATCATGAAGCCCCTTGAGCATCTGACTTCTGGCTAATAAAGG
AAATTTATTTTCATTGCAATAGTGTGTTGGAATTTTTTGTGTCTCTCACTCGGAAGGACA
TATGGGAGGGCAAATCATTTAAAACATCAGAATGAGTATTTGGTTTAGAGTTTGGCAA
CATATGCCCATTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGGGA
TAACGCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAATC
GACGCTCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATACCTGTC
CGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTT
ATCGCCACTGGCAGCAGCCACTGGTAACAGGATTAGCAGAGCGAGGTATGTAGGCGGT
GCTACAGAGTTCTTGAAGTGGTGGCCTAACTACGGCTACACTAGAAGAACAGTATTTG
GTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCG
CAGAAAAAAAGGATCTCAAGAAGATCCTTTGATCTTTTCTACGGGGTCTGACGCTCAGT
GGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTA
TTTCGTTCATCCATAGTTGCCTGACTCGGGGGGGGGGGGCGCTGAGGTCTGCCTCGTGA
AGAAGGTGTTGCTGACTCATACCAGGCCTGAATCGCCCCATCATCCAGCCAGAAAGTG
AGGGAGCCACGGTTGATGAGAGCTTTGTTGTAGGTGGACCAGTTGGTGATTTTGAACTT
TTGCTTTGCCACGGAACGGTCTGCGTTGTCGGGAAGATGCGTGATCTGATCCTTCAACT
CAGCAAAAGTTCGATTTATTCAACAAAGCCGCCGTCCCGTCAAGTCAGCGTAATGCTCT
GCCAGTGTTACAACCAATTAACCAATTCTGATTAGAAAAACTCATCGAGCATCAAATG
AAACTGCAATTTATTCATATCAGGATTATCAATACCATATTTTTGAAAAAGCCGTTTCT
GTAATGAAGGAGAAAACTCACCGAGGCAGTTCCATAGGATGGCAAGATCCTGGTATCG
GTCTGCGATTCCGACTCGTCCAACATCAATACAACCTATTAATTTCCCCTCGTCAAAAA
TAAGGTTATCAAGTGAGAAATCACCATGAGTGACGACTGAATCCGGTGAGAATGGCAA
AAGCTTATGCATTTCTTTCCAGACTTGTTCAACAGGCCAGCCATTACGCTCGTCATCAA
AATCACTCGCATCAACCAAACCGTTATTCATTCGTGATTGCGCCTGAGCGAGACGAAAT
ACGCGATCGCTGTTAAAAGGACAATTACAAACAGGAATCGAATGCAACCGGCGCAGG
AACACTGCCAGCGCATCAACAATATTTTCACCTGAATCAGGATATTCTTCTAATACCTG
GAATGCTGTTTTCCCGGGGATCGCAGTGGTGAGTAACCATGCATCATCAGGAGTACGG
ATAAAATGCTTGATGGTCGGAAGAGGCATAAATTCCGTCAGCCAGTTTAGTCTGACCAT
CTCATCTGTAACATCATTGGCAACGCTACCTTTGCCATGTTTCAGAAACAACTCTGGCG
CATCGGGCTTCCCATACAATCGATAGATTGTCGCACCTGATTGCCCGACATTATCGCGA
GCCCATTTATACCCATATAAATCAGCATCCATGTTGGAATTTAATCGCGGCCTCGAGCA
AGACGTTTCCCGTTGAATATGGCTCATAACACCCCTTGTATTACTGTTTATGTAAGCAG
ACAGTTTTATTGTTCATGATGATATATTTTTATCTTGTGCAATGTAACATCAGAGATTTT
GAGACACAACGTGGCTTTCCCCCCCCCCCCATTATTGAAGCATTTATCAGGGTTATTGT
CTCATGAGCGGATACATATTTGAATGTATTTAGAAAAATAAACAAATAGGGGTTCCGC
GCACATTTCCCCGAAAAGTGCCACCTGACGTCTAAGAAACCATTATTATCATGACATTA
ACCTATAAAAATAGGCGTATCACGAGGCCCTTTCGTCTCGCGCGTTTCGGTGATGACGG
TGAAAACCTCTGACACATGCAGCTCCCGGAGACGGTCACAGCTTGTCTGTAAGCGGAT
GCCGGGAGCAGACAAGCCCGTCAGGGCGCGTCAGCGGGTGTTGGCGGGTGTCGGGGCT
GGCTTAACTATGCGGCATCAGAGCAGATTGTACTGAGAGTGCACCATATGCGGTGTGA
AATACCGCACAGATGCGTAAGGAGAAAATACCGCATCAGATTGGCTAT

In certain embodiments, bi-shRNA-MSLN-2 comprises the following sequences:

(SEQ ID NO: 4)
Sense sequence: 5′ CCAGGACCAGCAGGAGGCA
(SEQ ID NO: 7)
Antisense sequence: 5′TGCCTCCTGCTGGTCCTGG

The insert for bi-shRNA-MSLN-2 vector is the following:

(SEQ ID NO: 8)
TCGACTGCTGTTGAAGTGAGCGCCCCAGGACCAGCAGGAGGCATAGTGAAGCC
ACAGATGTATGCCTCCTGCTGGTCCTGGGTTGCCTACTGCCTCGGAAGCAGCTCACTAC
ATTACTCAGCTGTTGAAGTGAGCGCCCCAGGACCTAGAGGAGTCATAGTGAAGCCACA
GATGTATGCCTCCTGCTGGTCCTGGGTTGCCTACTGCCTCGGAAGCTTAATAAAGGATC
TTTTATTTTCATTGGC

The full sequence for the vector pGBI-34 (bi-shRNA-MSLN-2) is the following:

(SEQ ID NO: 10)
TGGCCATTGCATACGTTGTATCCATATCATAATATGTACATTTATATTGGCTCAT
GTCCAACATTACCGCCATGTTGACATTGATTATTGACTAGTTATTAATAGTAATCAATT
ACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATG
TTCCCATAGTAACGCCAATAGGGACTTTCCATTGACGTCAATGGGTGGAGTATTTACGG
TAAACTGCCCACTTGGCAGTACATCAAGTGTATCATATGCCAAGTACGCCCCCTATTGA
CGTCAATGACGGTAAATGGCCCGCCTGGCATTATGCCCAGTACATGACCTTATGGGACT
TTCCTACTTGGCAGTACATCTACGTATTAGTCATCGCTATTACCATGGTGATGCGGTTTT
GGCAGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGT
CTATATAAGCAGAGCTCGTTTAGTGAACCGTCAGATCGCCTGGAGACGCCATCCACGCT
GTTTTGACCTCCATAGAAGACACCGGGACCGATCCAGCCTCCGCGGCCGGGAACGGTG
CATTGGAACGCGGATTCCCCGTGCCAAGAGTGACGTAAGTACCGCCTATAGACTCTAT
AGGCACACCCCTTTGGCTCTTATGCATGCTATACTGTTTTTGGCTTGGGGCCTATACACC
CCCGCTTCCTTATGCTATAGGTGATGGTATAGCTTAGCCTATAGGTGTGGGTTATTGAC
CATTATTGACCACTCCAACGGTGGAGGGCAGTGTAGTCTGAGCAGTACTCGTTGCTGCC
GCGCGCGCCACCAGACATAATAGCTGACAGACTAACAGACTGTTCCTTTCCATGGGTCT
TTTCTGCAGTCACCGTCGTCGACTGCTGTTGAAGTGAGCGCCCCAGGACCAGCAGGAG
GCATAGTGAAGCCACAGATGTATGCCTCCTGCTGGTCCTGGGTTGCCTACTGCCTCGGA
AGCAGCTCACTACATTACTCAGCTGTTGAAGTGAGCGCCCCAGGACCTAGAGGAGTCA
TAGTGAAGCCACAGATGTATGCCTCCTGCTGGTCCTGGGTTGCCTACTGCCTCGGAAGC
TTAATAAAGGATCTTTTATTTTCATTGGCGGCCGCGGATCCAGATCTTTTTCCCTCTGCC
AAAAATTATGGGGACATCATGAAGCCCCTTGAGCATCTGACTTCTGGCTAATAAAGGA
AATTTATTTTCATTGCAATAGTGTGTTGGAATTTTTTGTGTCTCTCACTCGGAAGGACAT
ATGGGAGGGCAAATCATTTAAAACATCAGAATGAGTATTTGGTTTAGAGTTTGGCAAC
ATATGCCCATTCTTCCGCTTCCTCGCTCACTGACTCGCTGCGCTCGGTCGTTCGGCTGCG
GCGAGCGGTATCAGCTCACTCAAAGGCGGTAATACGGTTATCCACAGAATCAGGGGAT
AACGCAGGAAAGAACATGTGAGCAAAAGGCCAGCAAAAGGCCAGGAACCGTAAAAAG
GCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCGTTCAGCCCGA
CCGCTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGTGC
TACAGAGTTCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTCTTGATCCGG
CAAACAAACCACCGCTGGTAGCGGTGGTTTTTTTGTTTGCAAGCAGCAGATTACGCGCA
GAAAAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATT
TCGTTCATCCATAGTTGCCTGACTCGGGGGGGGGGGGCGCTGAGGTCTGCCTCGTGAA
GAAGGTGTTGCTGACTCATACCAGGCCTGAATCGCCCCATCATCCAGCCAGAAAGTGA
GGGAGCCACGGTTGATGAGAGCTTTGTTGTAGGTGGACCAGTTGGTGATTTTGAACTTT
TGCTTTGCCACGGAACGGTCTGCGTTGTCGGGAAGATGCGTGATCTGATCCTTCAACTC
AGCAAAAGTTCGATTTATTCAACAAAGCCGCCGTCCCGTCAAGTCAGCGTAATGCTCTG
CCAGTGTTACAACCAATTAACCAATTCTGATTAGAAAAACTCATCGAGCATCAAATGA
AACTGCAATTTATTCATATCAGGATTATCAATACCATATTTTTGAAAAAGCCGTTTCTGT
AATGAAGGAGAAAACTCACCGAGGCAGTTCCATAGGATGGCAAGATCCTGGTATCGGT
CTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAA
GCTTATGCATTTCTTTCCAGACTTGTTCAACAGGCCAGCCATTACGCTCGTCATCAAAA
TCACTCGCATCAACCAAACCGTTATTCATTCGTGATTGCGCCTGAGCGAGACGAAATAC
GCGATCGCTGTTAAAAGGACAATTACAAACAGGAATCGAATGCAACCGGCGCAGGAA
CACTGCCAGCGCATCAACAATATTTTCACCTGAATCAGGATATTCTTCTAATACCTGGA
ATGCTGTTTTCCCGGGGATCGCAGTGGTGAGTAACCATGCATCATCAGGAGTACGGAT
AAAATGCTTGATGGTCGGAAGAGGCATAAATTCCGTCAGCCAGTTTAGTCTGACCATCT
CATCTGTAACATCATTGGCAACGCTACCTTTGCCATGTTTCAGAAACAACTCTGGCGCA
TCGGGCTTCCCATACAATCGATAGATTGTCGCACCTGATTGCCCGACATTATCGCGAGC
CCATTTATACCCATATAAATCAGCATCCATGTTGGAATTTAATCGCGGCCTCGAGCAAG
ACGTTTCCCGTTGAATATGGCTCATAACACCCCTTGTATTACTGTTTATGTAAGCAGAC
AGTTTTATTGTTCATGATGATATATTTTTATCTTGTGCAATGTAACATCAGAGATTTTGA
GACACAACGTGGCTTTCCCCCCCCCCCCATTATTGAAGCATTTATCAGGGTTATTGTCTC
ATGAGCGGATACATATTTGAATGTATTTAGAAAAATAAACAAATAGGGGTTCCGCGCA
CATTTCCCCGAAAAGTGCCACCTGACGTCTAAGAAACCATTATTATCATGACATTAACC
TATAAAAATAGGCGTATCACGAGGCCCTTTCGTCTCGCGCGTTTCGGTGATGACGGTGA
AAACCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGCTGGC
TTAACTATGCGGCATCAGAGCAGATTGTACTGAGAGTGCACCATATGCGGTGTGAAAT
ACCGCACAGATGCGTAAGGAGAAAATACCGCATCAGATTGGCTAT

### Sequence Listing

(SEQ ID NO: 1)
AGAGCTACCGGTGGACCCACGGTGCCTCCCTCCCTGGGATCTACACAGACCATG
GCCTTGCCAACGGCTCGACCCCTGTTGGGGTCCTGTGGGACCCCCGCCCTCGGCAGCCT
CCTGTTCCTGCTCTTCAGCCTCGGATGGGTGCAGCCCTCGAGGACCCTGGCTGGAGAGA
CAGGGCAGGAGGCTGCGCCCCTGGACGGAGTCCTGGCCAACCCACCTAACATTTCCAG
CCTCTCCCCTCGCCAACTCCTTGGCTTCCCGTGTGCGGAGGTGTCCGGCCTGAGCACGG
AGCGTGTCCGGGAGCTGGCTGTGGCCTTGGCACAGAAGAATGTCAAGCTCTCAACAGA
GCAGCTGCGCTGTCTGGCTCACCGGCTCTCTGAGCCCCCCGAGGACCTGGACGCCCTCC
CATTGGACCTGCTGCTATTCCTCAACCCAGATGCGTTCTCGGGGCCCCAGGCCTGCACC
CGTTTCTTCTCCCGCATCACGAAGGCCAATGTGGACCTGCTCCCGAGGGGGGCTCCCGA
GCGACAGCGGCTGCTGCCTGCGGCTCTGGCCTGCTGGGGTGTGCGGGGGTCTCTGCTGA
GCGAGGCTGATGTGCGGGCTCTGGGAGGCCTGGCTTGCGACCTGCCTGGGCGCTTTGTG
GCCGAGTCGGCCGAAGTGCTGCTACCCCGGCTGGTGAGCTGCCCGGGACCCCTGGACC
AGGACCAGCAGGAGGCAGCCAGGGCGGCTCTGCAGGGCGGGGGACCCCCCTACGGCC
CCCCGTCGACATGGTCTGTCTCCACGATGGACGCTCTGCGGGGCCTGCTGCCCGTGCTG
GGCCAGCCCATCATCCGCAGCATCCCGCAGGGCATCGTGGCCGCGTGGCGGCAACGCT
CCTCTCGGGACCCATCCTGGCGGCAGCCTGAACGGACCATCCTCCGGCCGCGGTTCCGG
CGGGAAGTGGAGAAGACAGCCTGTCCTTCAGGCAAGAAGGCCCGCGAGATAGACGAG
AGCCTCATCTTCTACAAGAAGTGGGAGCTGGAAGCCTGCGTGGATGCGGCCCTGCTGG
CCACCCAGATGGACCGCGTGAACGCCATCCCCTTCACCTACGAGCAGCTGGACGTCCT
AAAGCATAAACTGGATGAGCTCTACCCACAAGGTTACCCCGAGTCTGTGATCCAGCAC
CTGGGCTACCTCTTCCTCAAGATGAGCCCTGAGGACATTCGCAAGTGGAATGTGACGTC
CCTGGAGACCCTGAAGGCTTTGCTTGAAGTCAACAAAGGGCACGAAATGAGTCCTCAG
GTGGCCACCCTGATCGACCGCTTTGTGAAGGGAAGGGGCCAGCTAGACAAAGACACCC
TAGACACCCTGACCGCCTTCTACCCTGGGTACCTGTGCTCCCTCAGCCCCGAGGAGCTG
AGCTCCGTGCCCCCCAGCAGCATCTGGGCGGTCAGGCCCCAGGACCTGGACACGTGTG
ACCCAAGGCAGCTGGACGTCCTCTATCCCAAGGCCCGCCTTGCTTTCCAGAACATGAAC
GGGTCCGAATACTTCGTGAAGATCCAGTCCTTCCTGGGTGGGGCCCCCACGGAGGATTT
GAAGGCGCTCAGTCAGCAGAATGTGAGCATGGACTTGGCCACGTTCATGAAGCTGCGG
ACGGATGCGGTGCTGCCGTTGACTGTGGCTGAGGTGCAGAAACTTCTGGGACCCCACG
TGGAGGGCCTGAAGGCGGAGGAGCGGCACCGCCCGGTGCGGGACTGGATCCTACGGC
AGCGGCAGGACGACCTGGACACGCTGGGGCTGGGGCTACAGGGCGGCATCCCCAACG
GCTACCTGGTCCTAGACCTCAGCATGCAAGAGGCCCTCTCGGGGACGCCCTGCCTCCTA
GGACCTGGACCTGTTCTCACCGTCCTGGCACTGCTCCTAGCCTCCACCCTGGCCTGAGG
GCCCCACTCCCTTGCTGGCCCCAGCCCTGCTGGGGATCCCCGCCTGGCCAGGAGCAGGC
ACGGGTGGTCCCCGTTCCACCCCAAGAGAACTCGCGCTCAGTAAACGGGAACATGCCC
CCTGCAGACACGT
(SEQ ID NO: 2)
ATGGCCTTGCCAACGGCTCGACCCCTGTTGGGGTCCTGTGGGACCCCCGCCCTC
GGCAGCCTCCTGTTCCTGCTCTTCAGCCTCGGATGGGTGCAGCCCTCGAGGACCCTGGC
TGGAGAGACAGGGCAGGAGGCTGCGCCCCTGGACGGAGTCCTGGCCAACCCACCTAAC
ATTTCCAGCCTCTCCCCTCGCCAACTCCTTGGCTTCCCGTGTGCGGAGGTGTCCGGCCTG
AGCACGGAGCGTGTCCGGGAGCTGGCTGTGGCCTTGGCACAGAAGAATGTCAAGCTCT
CAACAGAGCAGCTGCGCTGTCTGGCTCACCGGCTCTCTGAGCCCCCCGAGGACCTGGA
CGCCCTCCCATTGGACCTGCTGCTATTCCTCAACCCAGATGCGTTCTCGGGGCCCCAGG
CCTGCACCCGTTTCTTCTCCCGCATCACGAAGGCCAATGTGGACCTGCTCCCGAGGGGG
GCTCCCGAGCGACAGCGGCTGCTGCCTGCGGCTCTGGCCTGCTGGGGTGTGCGGGGGT
CTCTGCTGAGCGAGGCTGATGTGCGGGCTCTGGGAGGCCTGGCTTGCGACCTGCCTGG
GCGCTTTGTGGCCGAGTCGGCCGAAGTGCTGCTACCCCGGCTGGTGAGCTGCCCGGGA
CCCCTGGACCAGGACCAGCAGGAGGCAGCCAGGGCGGCTCTGCAGGGCGGGGGACCC
CCCTACGGCCCCCCGTCGACATGGTCTGTCTCCACGATGGACGCTCTGCGGGGCCTGCT
GCCCGTGCTGGGCCAGCCCATCATCCGCAGCATCCCGCAGGGCATCGTGGCCGCGTGG
CGGCAACGCTCCTCTCGGGACCCATCCTGGCGGCAGCCTGAACGGACCATCCTCCGGC
CGCGGTTCCGGCGGGAAGTGGAGAAGACAGCCTGTCCTTCAGGCAAGAAGGCCCGCGA
GATAGACGAGAGCCTCATCTTCTACAAGAAGTGGGAGCTGGAAGCCTGCGTGGATGCG
GCCCTGCTGGCCACCCAGATGGACCGCGTGAACGCCATCCCCTTCACCTACGAGCAGCT
GGACGTCCTAAAGCATAAACTGGATGAGCTCTACCCACAAGGTTACCCCGAGTCTGTG
ATCCAGCACCTGGGCTACCTCTTCCTCAAGATGAGCCCTGAGGACATTCGCAAGTGGA
ATGTGACGTCCCTGGAGACCCTGAAGGCTTTGCTTGAAGTCAACAAAGGGCACGAAAT
GAGTCCTCAGGTGGCCACCCTGATCGACCGCTTTGTGAAGGGAAGGGGCCAGCTAGAC
AAAGACACCCTAGACACCCTGACCGCCTTCTACCCTGGGTACCTGTGCTCCCTCAGCCC
CGAGGAGCTGAGCTCCGTGCCCCCCAGCAGCATCTGGGCGGTCAGGCCCCAGGACCTG
GACACGTGTGACCCAAGGCAGCTGGACGTCCTCTATCCCAAGGCCCGCCTTGCTTTCCA
GAACATGAACGGGTCCGAATACTTCGTGAAGATCCAGTCCTTCCTGGGTGGGGCCCCC
ACGGAGGATTTGAAGGCGCTCAGTCAGCAGAATGTGAGCATGGACTTGGCCACGTTCA
TGAAGCTGCGGACGGATGCGGTGCTGCCGTTGACTGTGGCTGAGGTGCAGAAACTTCT
GGGACCCCACGTGGAGGGCCTGAAGGCGGAGGAGCGGCACCGCCCGGTGCGGGACTG
GATCCTACGGCAGCGGCAGGACGACCTGGACACGCTGGGGCTGGGGCTACAGGGCGG
CATCCCCAACGGCTACCTGGTCCTAGACCTCAGCATGCAAGAGGCCCTCTCGGGGACG
CCCTGCCTCCTAGGACCTGGACCTGTTCTCACCGTCCTGGCACTGCTCCTAGCCTCCACC
CTGGCCTGA
(SEQ ID NO: 3)
GCCTCATCTTCTACAAGAA
(SEQ ID NO: 4)
CCAGGACCAGCAGGAGGCA
(SEQ ID NO: 5)
TTCTTGTAGAAGATGAGGC
(SEQ ID NO: 6)
TCGACTGCTGTTGAAGTGAGCGCCGCCTCATCTTCTACAAGAATAGTGAAGCCA
CAGATGTATTCTTGTAGAAGATGAGGCGTTGCCTACTGCCTCGGAAGCAGCTCACTACA
TTACTCAGCTGTTGAAGTGAGCGCCGCCTCATCGAATACAAGAATAGTGAAGCCACAG
ATGTATTCTTGTAGAAGATGAGGCGTTGCCTACTGCCTCGGAAGCTTAATAAAGGATCT
TTTATTTTCATTGGC
(SEQ ID NO: 7)
TGCCTCCTGCTGGTCCTGG
(SEQ ID NO: 8)
TCGACTGCTGTTGAAGTGAGCGCCCCAGGACCAGCAGGAGGCATAGTGAAGCC
ACAGATGTATGCCTCCTGCTGGTCCTGGGTTGCCTACTGCCTCGGAAGCAGCTCACTAC
ATTACTCAGCTGTTGAAGTGAGCGCCCCAGGACCTAGAGGAGTCATAGTGAAGCCACA
GATGTATGCCTCCTGCTGGTCCTGGGTTGCCTACTGCCTCGGAAGCTTAATAAAGGATC
TTTTATTTTCATTGGC
pGBI-33 (bi-shRNA-MSLN-1):
(SEQ ID NO: 9)
TGGCCATTGCATACGTTGTATCCATATCATAATATGTACATTTATATTGGCTCAT
GTCCAACATTACCGCCATGTTGACATTGATTATTGACTAGTTATTAATAGTAATCAATT
ACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATG
TTCCCATAGTAACGCCAATAGGGACTTTCCATTGACGTCAATGGGTGGAGTATTTACGG
TAAACTGCCCACTTGGCAGTACATCAAGTGTATCATATGCCAAGTACGCCCCCTATTGA
CGTCAATGACGGTAAATGGCCCGCCTGGCATTATGCCCAGTACATGACCTTATGGGACT
TTCCTACTTGGCAGTACATCTACGTATTAGTCATCGCTATTACCATGGTGATGCGGTTTT
GGCAGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGT
CTATATAAGCAGAGCTCGTTTAGTGAACCGTCAGATCGCCTGGAGACGCCATCCACGCT
GTTTTGACCTCCATAGAAGACACCGGGACCGATCCAGCCTCCGCGGCCGGGAACGGTG
CATTGGAACGCGGATTCCCCGTGCCAAGAGTGACGTAAGTACCGCCTATAGACTCTAT
AGGCACACCCCTTTGGCTCTTATGCATGCTATACTGTTTTTGGCTTGGGGCCTATACACC
CCCGCTTCCTTATGCTATAGGTGATGGTATAGCTTAGCCTATAGGTGTGGGTTATTGAC
CATTATTGACCACTCCAACGGTGGAGGGCAGTGTAGTCTGAGCAGTACTCGTTGCTGCC
GCGCGCGCCACCAGACATAATAGCTGACAGACTAACAGACTGTTCCTTTCCATGGGTCT
TTTCTGCAGTCACCGTCGTCGACTGCTGTTGAAGTGAGCGCCGCCTCATCTTCTACAAG
AATAGTGAAGCCACAGATGTATTCTTGTAGAAGATGAGGCGTTGCCTACTGCCTCGGA
AGCAGCTCACTACATTACTCAGCTGTTGAAGTGAGCGCCGCCTCATCGAATACAAGAA
TAGTGAAGCCACAGATGTATTCTTGTAGAAGATGAGGCGTTGCCTACTGCCTCGGAAG
CTTAATAAAGGATCTTTTATTTTCATTGGCGGCCGCGGATCCAGATCTTTTTCCCTCTGC
CAAAAATTATGGGGACATCATGAAGCCCCTTGAGCATCTGACTTCTGGCTAATAAAGG
AAATTTATTTTCATTGCAATAGTGTGTTGGAATTTTTTGTGTCTCTCACTCGGAAGGACA
TATGGGAGGGCAAATCATTTAAAACATCAGAATGAGTATTTGGTTTAGAGTTTGGCAA
CATATGCCCATTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGGGA
TAACGCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAATC
GACGCTCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATACCTGTC
CGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTT
ATCGCCACTGGCAGCAGCCACTGGTAACAGGATTAGCAGAGCGAGGTATGTAGGCGGT
GCTACAGAGTTCTTGAAGTGGTGGCCTAACTACGGCTACACTAGAAGAACAGTATTTG
GTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCG
CAGAAAAAAAGGATCTCAAGAAGATCCTTTGATCTTTTCTACGGGGTCTGACGCTCAGT
GGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTA
TTTCGTTCATCCATAGTTGCCTGACTCGGGGGGGGGGGGCGCTGAGGTCTGCCTCGTGA
AGAAGGTGTTGCTGACTCATACCAGGCCTGAATCGCCCCATCATCCAGCCAGAAAGTG
AGGGAGCCACGGTTGATGAGAGCTTTGTTGTAGGTGGACCAGTTGGTGATTTTGAACTT
TTGCTTTGCCACGGAACGGTCTGCGTTGTCGGGAAGATGCGTGATCTGATCCTTCAACT
CAGCAAAAGTTCGATTTATTCAACAAAGCCGCCGTCCCGTCAAGTCAGCGTAATGCTCT
GCCAGTGTTACAACCAATTAACCAATTCTGATTAGAAAAACTCATCGAGCATCAAATG
AAACTGCAATTTATTCATATCAGGATTATCAATACCATATTTTTGAAAAAGCCGTTTCT
GTAATGAAGGAGAAAACTCACCGAGGCAGTTCCATAGGATGGCAAGATCCTGGTATCG
GTCTGCGATTCCGACTCGTCCAACATCAATACAACCTATTAATTTCCCCTCGTCAAAAA
TAAGGTTATCAAGTGAGAAATCACCATGAGTGACGACTGAATCCGGTGAGAATGGCAA
AAGCTTATGCATTTCTTTCCAGACTTGTTCAACAGGCCAGCCATTACGCTCGTCATCAA
AATCACTCGCATCAACCAAACCGTTATTCATTCGTGATTGCGCCTGAGCGAGACGAAAT
ACGCGATCGCTGTTAAAAGGACAATTACAAACAGGAATCGAATGCAACCGGCGCAGG
AACACTGCCAGCGCATCAACAATATTTTCACCTGAATCAGGATATTCTTCTAATACCTG
GAATGCTGTTTTCCCGGGGATCGCAGTGGTGAGTAACCATGCATCATCAGGAGTACGG
ATAAAATGCTTGATGGTCGGAAGAGGCATAAATTCCGTCAGCCAGTTTAGTCTGACCAT
CTCATCTGTAACATCATTGGCAACGCTACCTTTGCCATGTTTCAGAAACAACTCTGGCG
CATCGGGCTTCCCATACAATCGATAGATTGTCGCACCTGATTGCCCGACATTATCGCGA
GCCCATTTATACCCATATAAATCAGCATCCATGTTGGAATTTAATCGCGGCCTCGAGCA
AGACGTTTCCCGTTGAATATGGCTCATAACACCCCTTGTATTACTGTTTATGTAAGCAG
ACAGTTTTATTGTTCATGATGATATATTTTTATCTTGTGCAATGTAACATCAGAGATTTT
GAGACACAACGTGGCTTTCCCCCCCCCCCCATTATTGAAGCATTTATCAGGGTTATTGT
CTCATGAGCGGATACATATTTGAATGTATTTAGAAAAATAAACAAATAGGGGTTCCGC
GCACATTTCCCCGAAAAGTGCCACCTGACGTCTAAGAAACCATTATTATCATGACATTA
ACCTATAAAAATAGGCGTATCACGAGGCCCTTTCGTCTCGCGCGTTTCGGTGATGACGG
TGAAAACCTCTGACACATGCAGCTCCCGGAGACGGTCACAGCTTGTCTGTAAGCGGAT
GCCGGGAGCAGACAAGCCCGTCAGGGCGCGTCAGCGGGTGTTGGCGGGTGTCGGGGCT
GGCTTAACTATGCGGCATCAGAGCAGATTGTACTGAGAGTGCACCATATGCGGTGTGA
AATACCGCACAGATGCGTAAGGAGAAAATACCGCATCAGATTGGCTAT
(SEQ ID NO: 10)
TGGCCATTGCATACGTTGTATCCATATCATAATATGTACATTTATATTGGCTCAT
GTCCAACATTACCGCCATGTTGACATTGATTATTGACTAGTTATTAATAGTAATCAATT
ACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATG
TTCCCATAGTAACGCCAATAGGGACTTTCCATTGACGTCAATGGGTGGAGTATTTACGG
TAAACTGCCCACTTGGCAGTACATCAAGTGTATCATATGCCAAGTACGCCCCCTATTGA
CGTCAATGACGGTAAATGGCCCGCCTGGCATTATGCCCAGTACATGACCTTATGGGACT
TTCCTACTTGGCAGTACATCTACGTATTAGTCATCGCTATTACCATGGTGATGCGGTTTT
GGCAGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGT
CTATATAAGCAGAGCTCGTTTAGTGAACCGTCAGATCGCCTGGAGACGCCATCCACGCT
GTTTTGACCTCCATAGAAGACACCGGGACCGATCCAGCCTCCGCGGCCGGGAACGGTG
CATTGGAACGCGGATTCCCCGTGCCAAGAGTGACGTAAGTACCGCCTATAGACTCTAT
AGGCACACCCCTTTGGCTCTTATGCATGCTATACTGTTTTTGGCTTGGGGCCTATACACC
CCCGCTTCCTTATGCTATAGGTGATGGTATAGCTTAGCCTATAGGTGTGGGTTATTGAC
CATTATTGACCACTCCAACGGTGGAGGGCAGTGTAGTCTGAGCAGTACTCGTTGCTGCC
GCGCGCGCCACCAGACATAATAGCTGACAGACTAACAGACTGTTCCTTTCCATGGGTCT
TTTCTGCAGTCACCGTCGTCGACTGCTGTTGAAGTGAGCGCCCCAGGACCAGCAGGAG
GCATAGTGAAGCCACAGATGTATGCCTCCTGCTGGTCCTGGGTTGCCTACTGCCTCGGA
AGCAGCTCACTACATTACTCAGCTGTTGAAGTGAGCGCCCCAGGACCTAGAGGAGTCA
TAGTGAAGCCACAGATGTATGCCTCCTGCTGGTCCTGGGTTGCCTACTGCCTCGGAAGC
TTAATAAAGGATCTTTTATTTTCATTGGCGGCCGCGGATCCAGATCTTTTTCCCTCTGCC
AAAAATTATGGGGACATCATGAAGCCCCTTGAGCATCTGACTTCTGGCTAATAAAGGA
AATTTATTTTCATTGCAATAGTGTGTTGGAATTTTTTGTGTCTCTCACTCGGAAGGACAT
ATGGGAGGGCAAATCATTTAAAACATCAGAATGAGTATTTGGTTTAGAGTTTGGCAAC
ATATGCCCATTCTTCCGCTTCCTCGCTCACTGACTCGCTGCGCTCGGTCGTTCGGCTGCG
GCGAGCGGTATCAGCTCACTCAAAGGCGGTAATACGGTTATCCACAGAATCAGGGGAT
AACGCAGGAAAGAACATGTGAGCAAAAGGCCAGCAAAAGGCCAGGAACCGTAAAAAG
GCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCGTTCAGCCCGA
CCGCTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGTGC
TACAGAGTTCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTCTTGATCCGG
CAAACAAACCACCGCTGGTAGCGGTGGTTTTTTTGTTTGCAAGCAGCAGATTACGCGCA
GAAAAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACT
TGGTCTGACAGTTACCAATGCTTAATCAGTGAGGCACCTATCTCAGCGATCTGTCTATT
TCGTTCATCCATAGTTGCCTGACTCGGGGGGGGGGGGCGCTGAGGTCTGCCTCGTGAA
GAAGGTGTTGCTGACTCATACCAGGCCTGAATCGCCCCATCATCCAGCCAGAAAGTGA
GGGAGCCACGGTTGATGAGAGCTTTGTTGTAGGTGGACCAGTTGGTGATTTTGAACTTT
TGCTTTGCCACGGAACGGTCTGCGTTGTCGGGAAGATGCGTGATCTGATCCTTCAACTC
AGCAAAAGTTCGATTTATTCAACAAAGCCGCCGTCCCGTCAAGTCAGCGTAATGCTCTG
CCAGTGTTACAACCAATTAACCAATTCTGATTAGAAAAACTCATCGAGCATCAAATGA
AACTGCAATTTATTCATATCAGGATTATCAATACCATATTTTTGAAAAAGCCGTTTCTGT
AATGAAGGAGAAAACTCACCGAGGCAGTTCCATAGGATGGCAAGATCCTGGTATCGGT
CTGCGATTCCGACTCGTCCAACATCAATACAACCTATTAATTTCCCCTCGTCAAAAATA
AGGTTATCAAGTGAGAAATCACCATGAGTGACGACTGAATCCGGTGAGAATGGCAAAA
GCTTATGCATTTCTTTCCAGACTTGTTCAACAGGCCAGCCATTACGCTCGTCATCAAAA
TCACTCGCATCAACCAAACCGTTATTCATTCGTGATTGCGCCTGAGCGAGACGAAATAC
GCGATCGCTGTTAAAAGGACAATTACAAACAGGAATCGAATGCAACCGGCGCAGGAA
CACTGCCAGCGCATCAACAATATTTTCACCTGAATCAGGATATTCTTCTAATACCTGGA
ATGCTGTTTTCCCGGGGATCGCAGTGGTGAGTAACCATGCATCATCAGGAGTACGGAT
AAAATGCTTGATGGTCGGAAGAGGCATAAATTCCGTCAGCCAGTTTAGTCTGACCATCT
CATCTGTAACATCATTGGCAACGCTACCTTTGCCATGTTTCAGAAACAACTCTGGCGCA
TCGGGCTTCCCATACAATCGATAGATTGTCGCACCTGATTGCCCGACATTATCGCGAGC
CCATTTATACCCATATAAATCAGCATCCATGTTGGAATTTAATCGCGGCCTCGAGCAAG
ACGTTTCCCGTTGAATATGGCTCATAACACCCCTTGTATTACTGTTTATGTAAGCAGAC
AGTTTTATTGTTCATGATGATATATTTTTATCTTGTGCAATGTAACATCAGAGATTTTGA
GACACAACGTGGCTTTCCCCCCCCCCCCATTATTGAAGCATTTATCAGGGTTATTGTCTC
ATGAGCGGATACATATTTGAATGTATTTAGAAAAATAAACAAATAGGGGTTCCGCGCA
CATTTCCCCGAAAAGTGCCACCTGACGTCTAAGAAACCATTATTATCATGACATTAACC
TATAAAAATAGGCGTATCACGAGGCCCTTTCGTCTCGCGCGTTTCGGTGATGACGGTGA
AAACCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGCTGGC
TTAACTATGCGGCATCAGAGCAGATTGTACTGAGAGTGCACCATATGCGGTGTGAAAT
ACCGCACAGATGCGTAAGGAGAAAATACCGCATCAGATTGGCTAT.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A bifunctional shRNAs capable of reducing an expression of a Mesothelin gene; wherein at least one target site sequence of the bifunctional RNA molecule is located within the Mesothelin gene, wherein the bifunctional RNA molecule is capable of activating a cleavage-dependent and a cleavage-independent RNA-induced silencing complex for reducing the expression level of Mesothelin.

2. The bifunctional shRNAs of claim 1, wherein the bifunctional shRNA comprises at least one sequence defined by SEQ ID NO: 6.

3. The bifunctional shRNAs of claim 1, wherein the bifunctional shRNA comprises at least one sequence defined by SEQ ID NO: 8.

4. The bifunctional shRNAs of claim 1, wherein at least one target site sequence is within a human Mesothelin gene cDNA sequence (SEQ ID NO: 1 or 2).

5. The bifunctional shRNAs of claim 1, wherein at least one target site sequence is SEQ ID NO: 3 or SEQ ID NO: 4.

6. An expression vector comprising:
a promoter; and
a nucleic acid insert operably linked to the promoter, wherein the insert encodes one or more shRNA capable of inhibiting an expression of at least one target gene that is a Mesothelin gene via RNA interference;
wherein the one or more shRNA comprise a bifunctional RNA molecule that activates a cleavage-dependent and a cleavage-independent RNA-induced silencing complex for reducing the expression level of Mesothelin.

7. The expression vector of claim 6, wherein the target gene sequence comprises SEQ ID NO: 1 or 2.

8. The expression vector of claim 6, wherein a sequence arrangement for the shRNA comprises a 5′ stem arm-19 nucleotide target, which is Mesothelin-TA-15 nucleotide loop-19 nucleotide target complementary sequence-3′stem arm-Spacer-5′ stem arm-19 nucleotide target variant-TA-15 nucleotide loop-19 nucleotide target complementary sequence-3′stem arm.

9. The expression vector of claim 6, wherein the nucleic acid insert comprises at least one sequence selected from SEQ ID NO: 6 or SEQ ID NO: 8.

10. The expression vector of claim 6, wherein at least one shRNA has a target site sequence that is within a Mesothelin gene cDNA sequence.

11. The expression vector of claim 6, selected from the group consisting of SEQ ID NO: 9 or SEQ ID NO: 10.

12. A therapeutic delivery system comprising:
a therapeutic agent carrier; and
an expression vector comprising a promoter and a nucleic acid insert operably linked to the promoter, the nucleic acid insert encoding one or more short hairpin RNA (shRNA) capable inhibiting an expression of a target gene sequence that is Mesothelin gene via RNA interference;
wherein the one or more shRNA comprise a bifunctional RNA molecule that activates a cleavage-dependent and a cleavage-independent RNA-induced silencing complex for reducing the expression level of Mesothelin.

13. The delivery system of claim 12, wherein the therapeutic agent carrier is a compacted DNA nanoparticle.

14. The delivery system of claim 13, wherein the DNA nanoparticle is compacted with one or more polycations.

15. The delivery system of claim 14, wherein the one or more polycations is a 10 kDA polyethylene glycol (PEG)-substituted cysteine-lysine 3-mer peptide (CK₃₀PEG10k).

16. The delivery system of claim 13, wherein the compacted DNA nanoparticles are further encapsulated in a liposome.

17. The delivery system of claim 16, wherein the liposome is a bilamellar invaginated vesicle (BIV).

18. The delivery system of claim 16, wherein the liposome is a reversibly masked liposome.

19. The delivery system of claim 12, wherein the therapeutic agent carrier is a liposome.

20. The delivery system of claim 12, wherein the target gene sequence comprises SEQ ID NO: 3 or 4.

21. The delivery system of claim 12, wherein the nucleic acid insert comprises at least one of the sequences selected from SEQ ID NO: 6, SEQ ID NO: 8, SEQ ID NO: 9, or SEQ ID NO: 10.

22. A method to deliver one or more shRNAs to a target tissue expressing a Mesothelin gene comprising the steps of:
preparing an expression vector comprising a promoter and a nucleic acid insert operably linked to the promoter that encodes the one or more shRNA, wherein the one or more shRNA comprise a bifunctional RNA molecule that activates a cleavage-dependent and a cleavage-independent RNA-induced silencing complex for reducing the expression level of Mesothelin;
combining the expression vector with a therapeutic agent carrier, wherein the therapeutic agent carrier comprises a liposome; and
administering a therapeutically effective amount of the expression vector and therapeutic agent carrier complex to a patient in need thereof.

23. The method of claim 22, wherein the therapeutic agent carrier is a compacted DNA nanoparticle.

24. The method of claim 23, wherein the DNA nanoparticle is compacted with one or more polycations, wherein the one or more polycations comprise a 10 kDA polyethylene glycol (PEG)-substituted cysteine-lysine 3-mer peptide (CK₃₀PEG10k) or a 30-mer lysine condensing peptide.

25. The method of claim 23, wherein the compacted DNA nanoparticles are further encapsulated in a liposome, wherein the liposome is a bilamellar invaginated vesicle (BIV) and is decorated with one or more “smart” receptor targeting moieties.

26. The method of claim 25, wherein the one or more “smart” receptor targeting moieties are small molecule bivalent beta-turn mimics.

27. The method of claim 25, wherein the liposome is a reversibly masked liposome.

28. The method of claim 22, wherein the liposome is a bilamellar invaginated vesicle (BIV).

29. The method of claim 22, wherein the liposome is a reversibly masked liposome.

30. The method of claim 22, wherein the one or more “smart” receptor targeting moieties are small molecule bivalent beta-turn mimics.

31. The method of claim 22, wherein the vector is selected from one of SEQ ID NO: 9 or SEQ ID NO: 10.

32. The method of claim 22, wherein the nucleic acid insert comprises a sequence selected from SEQ ID NO: 6, or SEQ ID NO: 8.

33. A method to inhibit an expression of a Mesothelin gene in one or more target cells comprising the steps of:
selecting the one or more target cells; and
transfecting the target cell with a vector that expresses one or more short hairpin RNA (shRNAs) capable of inhibiting an expression of a Mesothelin gene in the one or more target cells via RNA interference, wherein the one or more shRNA comprise a bifunctional RNA molecule that activates a cleavage-dependent and a cleavage-independent RNA-induced silencing complex for reducing the expression level of Mesothelin.

34. The method of claim 33, wherein the shRNA incorporates siRNA (cleavage-dependent) and miRNA (cleavage-independent) motifs.

35. A method of suppressing a tumor cell growth in a human subject comprising the steps of:
identifying the human subject in need for suppression of the tumor cell growth; and
administering an expression vector in a therapeutic agent carrier complex to the human subject in an amount sufficient to suppress the tumor cell growth, wherein the expression vector expresses one or more shRNA capable inhibiting an expression of a target gene that is Mesothelin in the one or more target cells via RNA interference;
wherein the one or more shRNA comprise a bifunctional RNA molecule that activates a cleavage-dependent and a cleavage-independent RNA-induced silencing complex for reducing the expression level of the target gene;
wherein the inhibition results in an apoptosis, an arrested proliferation, or a reduced invasiveness of the tumor cells.

36. The method of claim 35, wherein the therapeutic agent carrier comprises a bilamellar invaginated vesicle (BIV).

37. The method of claim 35, wherein the therapeutic agent carrier comprises one or more “smart” receptor targeting moieties are small molecule bivalent beta-turn mimics.

38. The method of claim 35, wherein administering is selected from the group consisting of subcutaneous, intravenous, intraperitoneal, intramuscular, and intravenous injection.

39. The method of claim 35, wherein administering comprises intratumoral injection.

40. The method of claim 35, wherein administering comprises injecting with a DNA:lipoplex.

41. The method of claim 35, wherein the tumor cell growth expresses Mesothelin.

42. The method of claim 35, wherein the tumor cell growth is human pancreatic ductal adenocarcinoma.

43. The method of claim 35, wherein the tumor cell growth is selected from the group consisting of insulinoma, mesothelioma, ovarian cancer, and pancreatic cancer.

44. The method of claim 35, wherein the tumor cell growth is selected from the group consisting of epithelial mesothelioma, squamous cell carcinoma, head and neck cancer, lung cancer, cervix cancer, esophagus cancer, and gastric adenocarcinoma.


SEQUENCE LISTING (10 sequences)

SEQ ID NO: 1 - DNA, 2052 bp, Homo sapiens
agagctaccggtggacccacggtgcctccctccctgggatctacacagaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccgtttcttctcccgcatcacgaaggccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcggggacgccctgcctcctaggacctggacctgttctcaccgtcctggcactgctcctagcctccaccctggcctgagggccccactcccttgctggccccagccctgctggggatccccgcctggccaggagcaggcacgggtggtccccgttccaccccaagagaactcgcgctcagtaaacgggaacatgccccctgcagacacgt

SEQ ID NO: 2 - DNA, 1869 bp, Homo sapiens
atggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccgtttcttctcccgcatcacgaaggccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcatgcaagaggccctctcggggacgccctgcctcctaggacctggacctgttctcaccgtcctggcactgctcctagcctccaccctggcctga

SEQ ID NO: 3 - DNA, 19 bp, Artificial Sequence
  Synthetic primer
gcctcatcttctacaagaa

SEQ ID NO: 4 - DNA, 19 bp, Artificial Sequence
  Synthetic primer
ccaggaccagcaggaggca

SEQ ID NO: 5 - DNA, 19 bp, Artificial Sequence
  Synthetic primer
ttcttgtagaagatgaggc

SEQ ID NO: 6 - DNA, 249 bp, Artificial Sequence
  Synthetic primer
tcgactgctgttgaagtgagcgccgcctcatcttctacaagaatagtgaagccacagatgtattcttgtagaagatgaggcgttgcctactgcctcggaagcagctcactacattactcagctgttgaagtgagcgccgcctcatcgaatacaagaatagtgaagccacaggradatgtattcttgtagaagatgaggcgttgcctactgcctcggaagcttaataaaggatcttttattttcattggc

SEQ ID NO: 7 - DNA, 19 bp, Artificial Sequence
  Synthetic primer
tgcctcctgctggtcctgg

SEQ ID NO: 8 - DNA, 245 bp, Artificial Sequence
  Synthetic primer
tcgactgctgttgaagtgagcgccccaggaccagcaggaggcatagtgaagccacagatgtatgcctcctgctggtcctgggttgcctactgcctcggaagcagctcactacattactcagctgttgaagtgagcgccccaggacctagaggagtcatagtgaagccacagatgtatgcctcctgctggtcctgggttgcctactgcctcggaagcttaataaaggatcttttattttcattggc

SEQ ID NO: 9 - DNA, 4221 bp, Homo sapiens
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgactgctgttgaagtgagcgccgcctcatcttctacaagaatagtgaagccacagatgtattcttgtagaagatgaggcgttgcctactgcctcggaagcagctcactacattactcagctgttgaagtgagcgccgcctcatcgaatacaagaagradtagtgaagccacagatgtattcttgtagaagatgaggcgttgcctactgcctcggaagcttaataaaggatcttttattttcattggcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaagradtaaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 10 - DNA, 4221 bp, Homo sapiens
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtgradctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgactgctgttgaagtgagcgccccaggaccagcaggaggcatagtgaagccacagatgtatgcctcctgctggtcctgggttgcctactgcctcggaagcagctcactacattactcagctgttgaagtgagcgccccaggacctagaggagtcatagtgaagccacagatgtatgcctcctgctggtcctgggttgcctactgcctcggaagcttaataaaggatcttttattttcattggcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaactgradtggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat